(19) United States
(12) Patent Application Publication
(10) Pub. No.: US 2024/0087160 A1
(43) Pub. Date: Mar. 14, 2024

(54) RECONSTRUCTIVE IMAGING OF INTERNAL BODY PARTS AND MOUTH

(71) Applicant: Ali Ebrahimi Afrouzi, Henderson, NV (US)

(72) Inventor: Ali Ebrahimi Afrouzi, Henderson, NV (US)

(73) Assignee: AI Incorporated, Toronto (CA)

(21) Appl. No.: 18/505,012

(22) Filed: Nov. 8, 2023

Related U.S. Application Data
(60) Provisional application No. 63/446,840, filed on Feb. 18, 2023.
(60) Provisional application No. 63/404,660, filed on Sep. 8, 2022.

(51) Int. Cl. G06T 7/73; G06T 7/246; G06T 7/50

(52) U.S. Cl. CPC G06T 7/73; G06T 7/246; G06T 7/50; A61B 1/2736; G06T 2207/10028; G06T 2207/10068; G06T 2207/30036

(57) ABSTRACT

A method for localizing an electronic device, including: capturing data of surroundings of the electronic device with at least one sensor of the electronic device; and inferring a location of the electronic device based on at least some of the data of the surroundings, wherein inferring the location of the electronic device includes: determining a probability of the electronic device being located at different possible locations within the surroundings based on the at least some of the data of the surroundings; and inferring the location of the electronic device based on the probability of the electronic device being located at different possible locations within the surroundings.

### CROSS REFERENCE TO RELATED APPLICATIONS

This application claims the benefit of U.S. Provisional Patent Application Nos. 63446840, filed Feb. 18, 2023, and 63/404,660, filed Sep. 8, 2022, each of which is hereby incorporated herein by reference.

In this patent, certain U.S. patents, U.S. patent applications, or other materials (e.g., articles) have been incorporated by reference. Specifically, U.S. patent application Ser. Nos. 16/109,617, 17/494,251, 17/344,892, 17/670,277, 17/990,743, 15/272,752, 17/878,725, and 18/503,093, 16/239,410, 17/693,946, 16/163,541, 16/048,185, 16/048,179, 16/920,328, 16/163,562, 16/724,328, 16/163,508, 16/163,541, 16/048,185, 16/048,179, 16/920,328, 16/163,562, 16/724,328, 16/163,508, 15/976,853, 15/442,992, 16/832,180, 17/403,292, 16/995,500, 15/447,122, 16/932,495, 15/257,798, 15/243,783, 15/954,410, 16/832,221, 15/224,442, 15/674,310, 15/683,255, 15/425,130, 15/955,480, 16/554,040, 15/425,130, 15/955,480, 16/554,040, 16/297,508, and Ser. No. 16/509,099 are hereby incorporated by reference. The text of such U.S. patents, U.S. patent applications, and other materials is, however, only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

### FIELD OF THE DISCLOSURE

The disclosure generally relates to electronic medical devices, and more particularly, to SLAM-enabled electronic medical devices.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented below.

A method for localizing an electronic device, including: capturing data of surroundings of the electronic device with at least one sensor of the electronic device; and inferring a location of the electronic device based on at least some of the data of the surroundings, wherein inferring the location of the electronic device includes: determining a probability of the electronic device being located at different possible locations within the surroundings based on the at least some of the data of the surroundings; and inferring the location of the electronic device based on the probability of the electronic device being located at different possible locations within the surroundings.

### BRIEF DESCRIPTION OF DRAWINGS

Steps shown in the figures may be modified, may include additional and/or omit steps in an actual implementation, and may be performed in a different order than shown in the figures. Further, the figures illustrated and described may be according to only some embodiments.

FIGS. 1 and 2 illustrate an example of a SLAM enabled endoscopic device.

FIGS. 3A-3D illustrate an example of an application of a communication device paired with the SLAM enabled endoscopic device.

FIG. 4 illustrates an example of a SLAM enabled scanning device.

FIGS. 5 and 6 illustrate an example of an application of a communication device paired with the SLAM enabled endoscopic device.

FIG. 7 illustrates an example of a reconstructed model of a mouth.

FIG. 8 illustrates an example of a person undergoing laparoscopic surgery.

FIG. 9 illustrates an example of a dummy surgical tool.

FIG. 10 illustrates using depth data and image data to generate a dynamic depth field.

FIGS. 11A-11G illustrate an example of a SLAM-enabled handheld device and a paired application of a communication device.

FIGS. 12A and 12 B illustrate an example of a SLAM-enabled handheld device.

FIGS. 13A and 13B illustrate examples of a module comprising at least one of a line laser and at least one of an image sensor.

FIGS. 14 and 15 illustrate examples of structured light and measuring distances using triangulation.

FIGS. 16 and 17 illustrate examples of TOF sensors for accurately measuring distance.

FIG. 18 illustrates changes in accuracy for distances derived through triangulation.

FIG. 19 illustrates a relation between confidence score and a location of transmitters and receivers.

FIGS. 20 and 21 illustrate an example of decomposition of affine transformations.

FIG. 22 illustrates a translation in 2D described as a shear in 3D.

FIG. 23 illustrates preservations of lines and parallelism in affine transformation.

FIG. 24 illustrates an example of a bounding volume of an object.

FIG. 25 illustrates feature extraction from images.

FIG. 26 illustrates combining sensor data channels.

FIGS. 27 and 29 illustrate an example of a process for localizing a medical device.

FIG. 30 illustrates a diagram of camera object and camera state vector extraction.

FIG. 31 illustrates a correlation between observability and computation needs.

FIG. 32 illustrates an example of a process of localizing a medical device.

FIG. 33 illustrates an example of an MCU and tasks executed by the MCU.

### DETAILED DESCRIPTION OF SOME EMBODIMENTS

Embodiments herein relate to electronic medical devices, and more particularly, to SLAM-enabled electronic medical devices. Examples of medical devices include a handheld scanning device, an endoscopic device, and other medical instruments or tools used by surgeons or other medical professionals, and in some cases, a consumer.

SLAM technology may be useful in reconstruction of 3D models of internal organs. 3D models of internal organs may help medical professionals better understand different medical situations of patients. Some aspects include a SLAM enabled system comprising a camera and at least one light source (similar to an endoscopic camera), in addition to a gyroscope and/or an IMU for tracking orientation and movement of the camera. As the camera moves within the body data is transmitted from the camera, gyroscope, IMU, etc. to an application executed on a communication device (e.g., laptop, desktop computer, tablet, smart phone). Similar to an endoscopic camera, an operator of the SLAM enabled system guides movement of the camera by moving the camera forwards, backwards, and rotationally, and adjusts the orientation of the camera. In some embodiments, the application generates a point cloud and a 3D model of tissues captured within the body based on images captured by the camera and movement data captured by the gyroscope and/or IMU. To generate the point cloud, the application track points of interest within the stream of images in real time. The application may combine the tracked points of interest within the stream of images with movement data to generate a more accurate representation of the tissues within the body. In some embodiments, the application guides the operator to move the camera in a particular direction or orientation to capture areas missing from a field of view of the camera. The application determines the particular direction and orientation required to capture the areas missing based on data captured by the camera, gyroscope, and/or IMU indicative of a current location and orientation of the camera.

Data indicative of movement and orientation of the camera and machine learning algorithms are important in the operation of the SLAM enabled system, as internal organs are flexible and constantly moving. In embodiments, the application distinguishes movement of the organs (i.e., environment) from movement of the camera and isolates and reconstructs a 3D model of the organs. Using SLAM techniques, the application predicts a best next position for the camera. In some embodiments, the application assists the operator in choosing movements and/or orientation of the camera. In some embodiments, the system actuates autonomous course correction of movements of the camera chosen by the operator or autonomously determines and actuates movements and/or orientation of the camera. In some cases, the SLAM enabled system may be used at home by everyday consumers to generate 3D models as the application provides guidance to the operator in the movements and orientation of the camera or actuates semi or full autonomous movement and orientation of the camera.

In some embodiments, the application is configured to display a camera view of the camera, suggested directions of movement of the camera within the body, and a control panel for guiding the camera. The suggested directions may be superimposed on the camera view. In some embodiments, the application is configured to display partial and full 3D reconstruction of internal organs, wherein the 3D reconstruction is gradually completed as the camera moves within the body and more data is captured. In some embodiments, the SLAM enabled system is used in dentistry or orthodontics for generating a 3D model of an interior of a mouth.

In some embodiments, the application uses machine learning to learn approximate positions of different internal organs and classification of internal organs. In some embodiments, machine learning may be used to help in diagnoses based on characteristics of particular internal organs (e.g., size, shape, abnormalities, etc.). Machine learning may similarly be used in dentistry and orthodontics, wherein machine learning is used to learn approximate positions of different teeth and classification of teeth. Machine learning may be used to help in detection of cavities, decaying teeth, gum issues, receding gum, broken teeth, etc. based on observed characteristics. In some embodiments, the SLAM enabled system further comprises a depth sensor and/or an ultrasound sensor to provide more data.

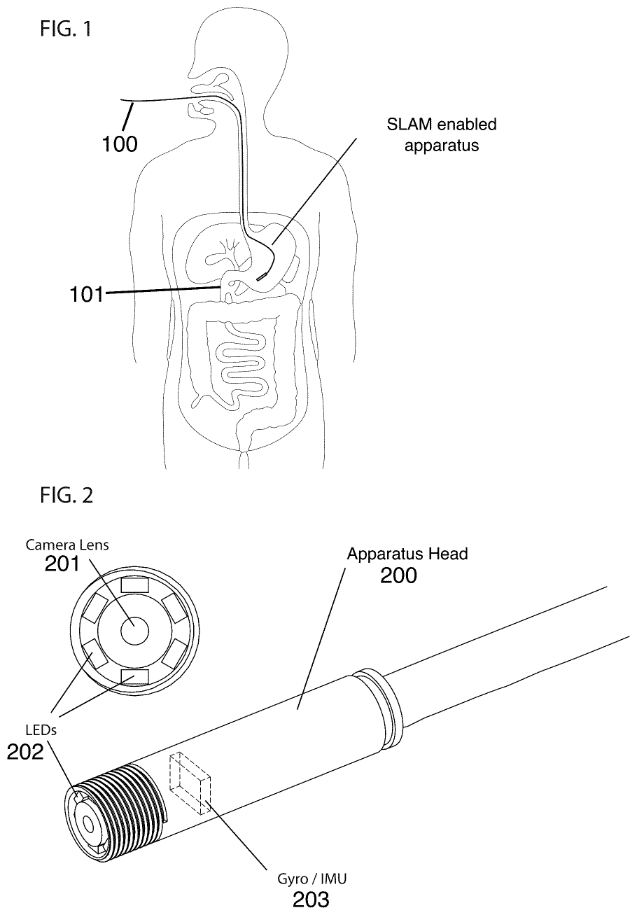

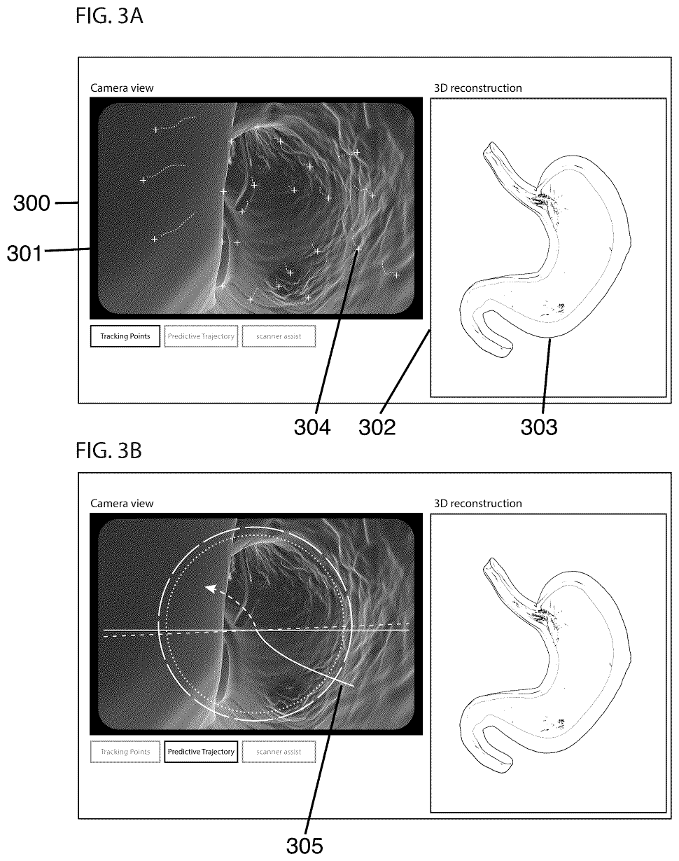

FIG. 1 illustrates an example of a SLAM enabled endoscopic device 100 within a digestive system of a body 101. FIG. 2 illustrates main components of the SLAM enabled endoscopic device comprising a head 200, a camera 201, LEDs 202, and gyroscope/IMU sensor 203. The camera 201 captures images within the body 101 and the gyroscope/IMU sensor 203 captures data indicative of movement and orientation of the camera 201. FIGS. 3A-3D illustrate an application 300 displaying a camera view 301 and a 3D reconstruction 302 of an internal organ 303. In FIGS. 3A, the camera view 301 displays tracking points 304. As the camera 201 moves around, various points of interest 304 are automatically detected and tracked. Using data captured by the gyroscope/IMU sensor 203, the application 300 determines position, orientation, and speed of movement of the camera, which the application 300 then uses in tracking the location of points of interest 304 in 3D space. The location of points of interest 304 are used by the application 300 to reconstruct the 3D model 303. In FIG. 3B, the application 300 displays a predictive trajectory 305 of the camera 201 to help an operator in moving the camera 201 within the body. In FIG. 3C, the application 300 displays a scanner assist, wherein fully scanned areas 306 are highlighted to inform the operator which areas need to be scanned next. In FIG. 3D, the application 300 displays a real time location 307 of the camera 201 and a camera field of view 308. This provides the operator with information on current observation and where to observe next.

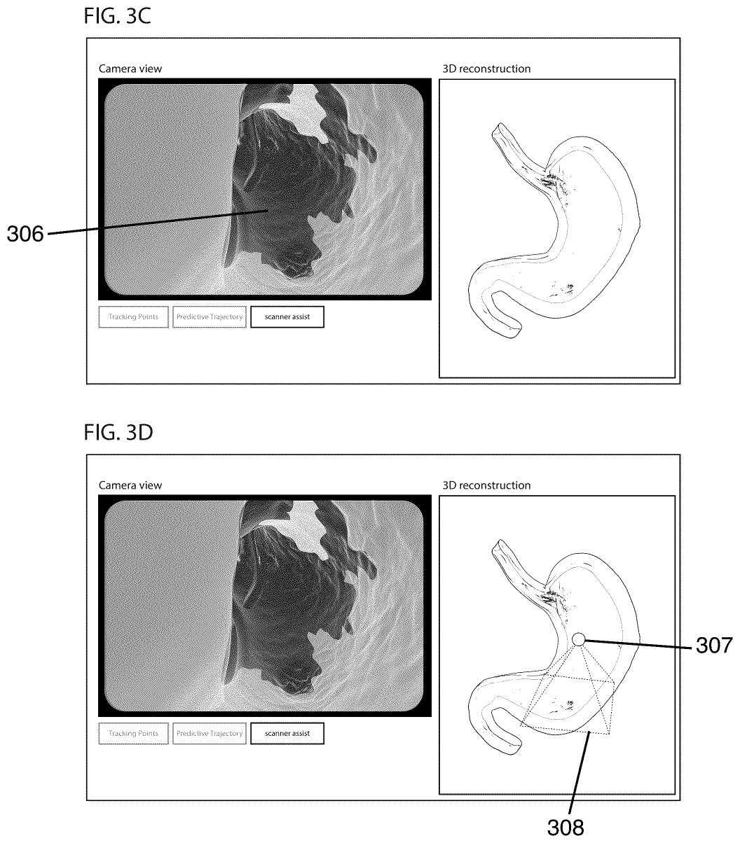

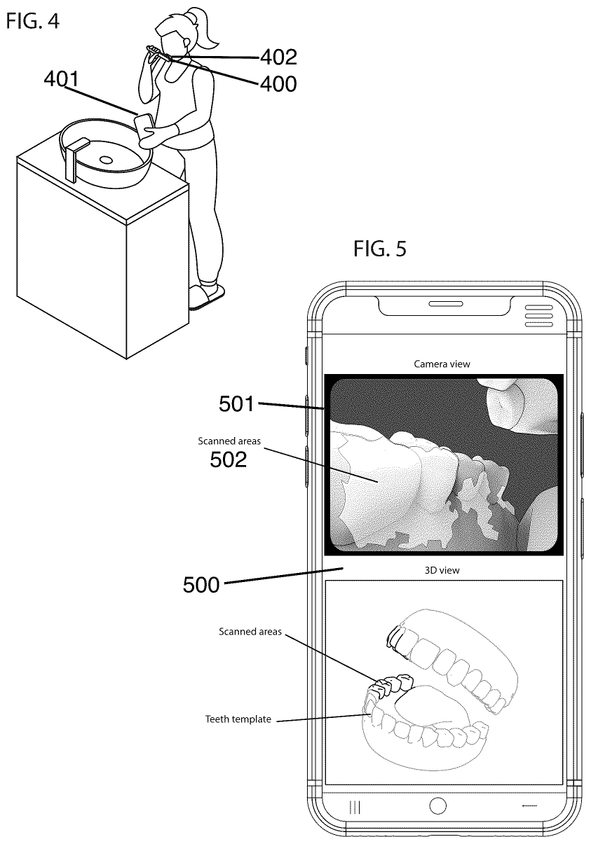

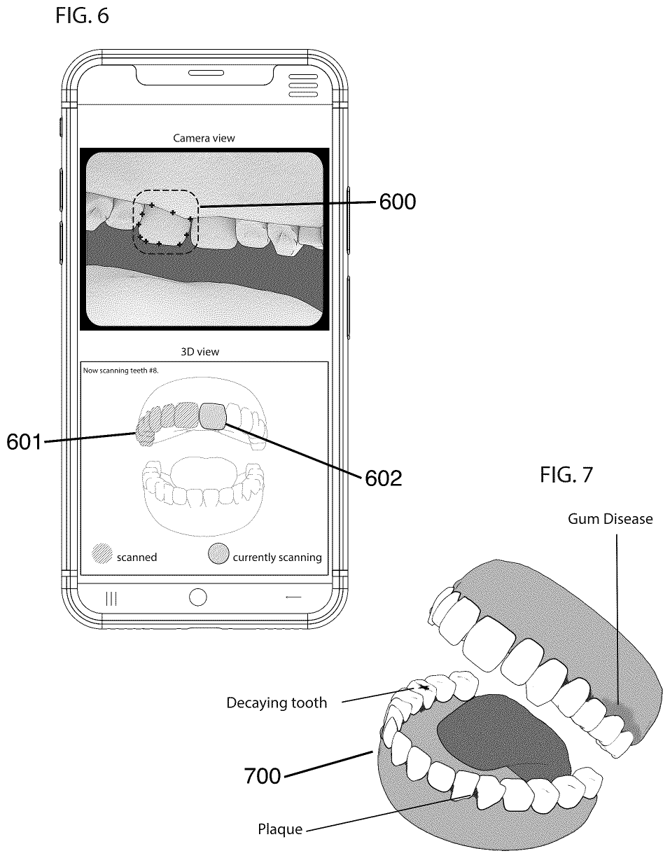

FIG. 4 illustrates a variation of a SLAM enabled system for everyday consumers (i.e., those without specialized skills in operating medical scanning equipment) comprising device 400 connected to an application of a communication device 401. The device 400 may be used to scan an interior of a mouth of a user 402. FIG. 5 illustrates the application 500 displaying a camera view 501 including scanned areas and 3D reconstruction 502 including scanned areas and a template of the teeth. Some embodiments may reconstruct the 3D model from scratch which may be provided as a continuous mesh of the entire mouth. In other embodiments, a template of the teeth may be displayed with each individual tooth labeled. By scanning the inside of the mouth, new data captured by sensors of the device 400 reshapes the 3D model of each tooth. Data pertaining to each tooth may be used by the application to analyze each individual tooth. FIG. 6 illustrates the application 500 guiding the user 402 in scanning each tooth individually, the camera view 501 highlighting the tooth of interest 600 and the 3D reconstruction 502 highlighting teeth scanned 601 and the current tooth being scanned 602. FIG. 7 illustrates the completed 3D reconstructed model 700 that may be used to extract more information than a traditional clay mold. The application uses visual clues, such as texture, shape, and color, to suggest possible diagnoses (e.g., decay, plaque, cavity, gum disease, etc.).

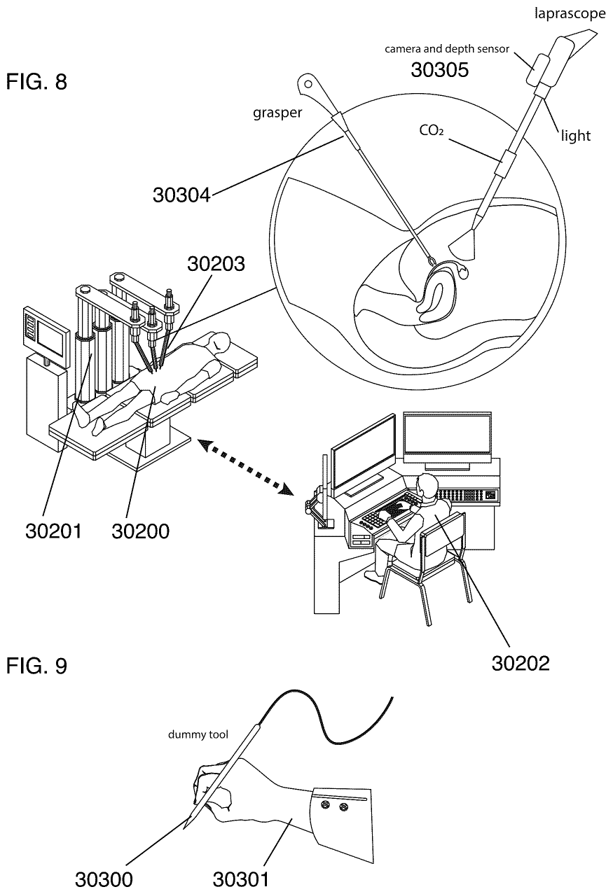

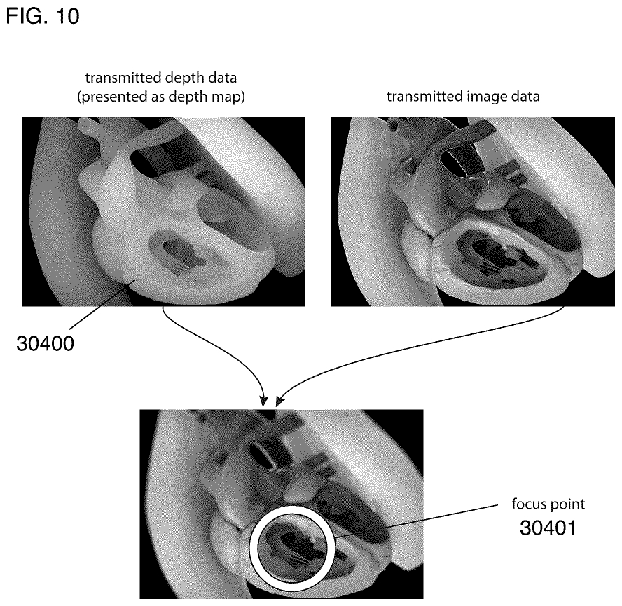

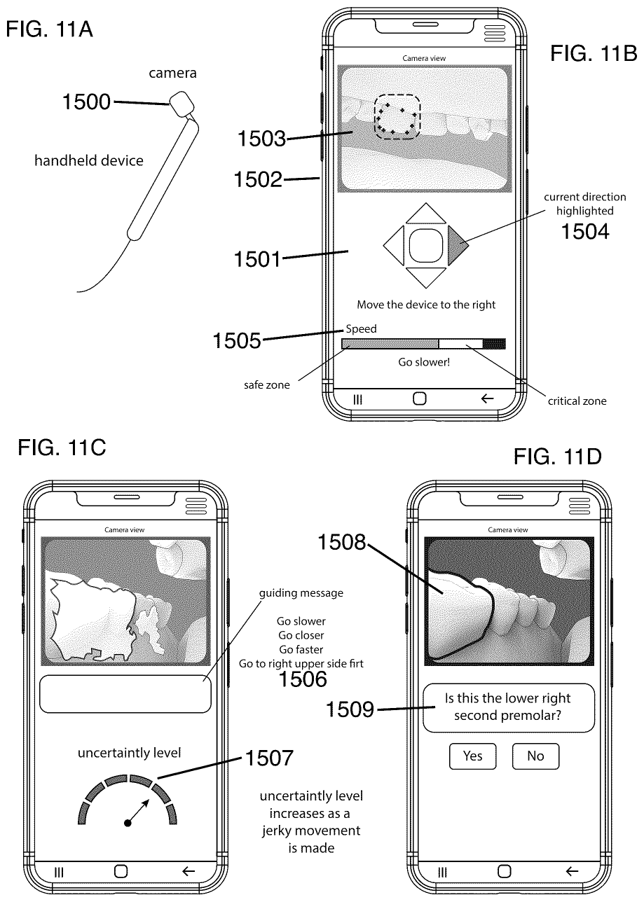

Another example includes using the robot for remote surgery on a patient. There may be several reasons for remote surgery, such as logistics (e.g., the surgeon and the patient cannot be in the same physical environment due to surgeon shortage, scheduling conflicts, high cost (time and monetary) of long distance travel, etc. or should not be in the same physical environment for safety reasons such as high risk of bacterial or viral infection, use of radiation, etc.) and scale of operation (e.g., the operation area is too small for direct operation). In the case of a small operation area, the movements of the robot are scaled down versions of the operator movements, wherein the operator sees the scaled version of what the robot sees. Using the robot, the size of an opened area on the patient body is smaller compared to when traditional methods are used as the robot arm and tools are smaller and more flexible. A smaller opening in the body of the patient may allow the patient to recover faster after surgery. Depending on the needed accuracy, the operator may use different types of interfaces to control the robot remotely. The operator may use a specific joystick or keyboard controller designed to move different robot arms with several buttons by engaging, disengaging, gripping, and releasing tissues, switching tools, and such. FIG. 8 illustrates a person 30200 undergoing laparoscopic surgery using a robot 30201 controlled remotely by a user 30202. Arms 30203 of the robot 30201 are used to operate on the user 30200 using tools 30204. The tools 30204 include a camera and depth sensor 30205 such that the user 30202 may continuously view the incision site and control the robot 30201 accordingly. Another method includes using a dummy tool as the main instrument. The dummy tool can transmit its position and orientation to the robot such that the robot aligns and positions its own arm (and the real tool) accordingly. This may help the operator perform their job more intuitively. The dummy tool may be equipped with haptic sensors and triggers to transmit the pressure applied to the tool of the robot and trigger the hand of the operator when the robot tool touches different surfaces. FIG. 9 illustrates an example of a dummy tool 30300 held by a user 30301. One major difference for this method of remote operation compared to current methods of telesurgery (or other remote operating methods) is the inclusion of depth data in the data stream. Since the robot can capture and transmit the depth data in combination with image data, the operator has a better understanding of the situation in the remote area. The depth data may be presented as a separate image stream showing the order of layers and far to near objects in a grayscale format or in different hues. Alternatively, the depth data may be used to enhance the image stream itself by applying digital depth of field to the image. For example, a location of the tip of the current surgery tool is defined as the focus point and the area surrounding it may become defocused as the depth shifts from that point. Or in more sophisticated embodiments, the display system may be equipped with an eye tracker (e.g., implemented on the monitor, or the on VR, AR, XR headsets) and the area that the operator is observing is defined as the focus point. FIG. 10 illustrates using both depth data and image data to generate a dynamic depth field 30400, with the area of interest 30401 in focus.

In embodiments, images of a stream of images are marked with a pose, orientation, and/or coordinate of the image sensor from which the images were recorded. Such information (e.g., pose, orientation, coordinate, etc.) may be captured using data from the same image sensor. For example, pose, orientation, and/or coordinate of an image sensor may be captured by tracking features in previous images captured by the image sensor, using an image sensor with a laser grid point that shines at intervals, and by tracking currently observed features in images captured by the image sensor and comparing them against a previously generated mapping. In other cases, pose, orientation, and/or coordinate of the image sensor may be captured using a secondary sensor, such as GPS used in outdoor settings, a LIDAR used in indoor or outdoor settings, and an IMU used in hand held devices or wearables.

In some applications, such as the mapping of internal body parts (e.g., using endoscopic devices) or mouth reconstruction, only relative change in pose may be needed. In some embodiments, capturing pose information that is attached to the coordinate of the capturing device is only required. In some embodiments, pose information in the coordinate system of the capturing device is transformed into the coordinate system of an environment.

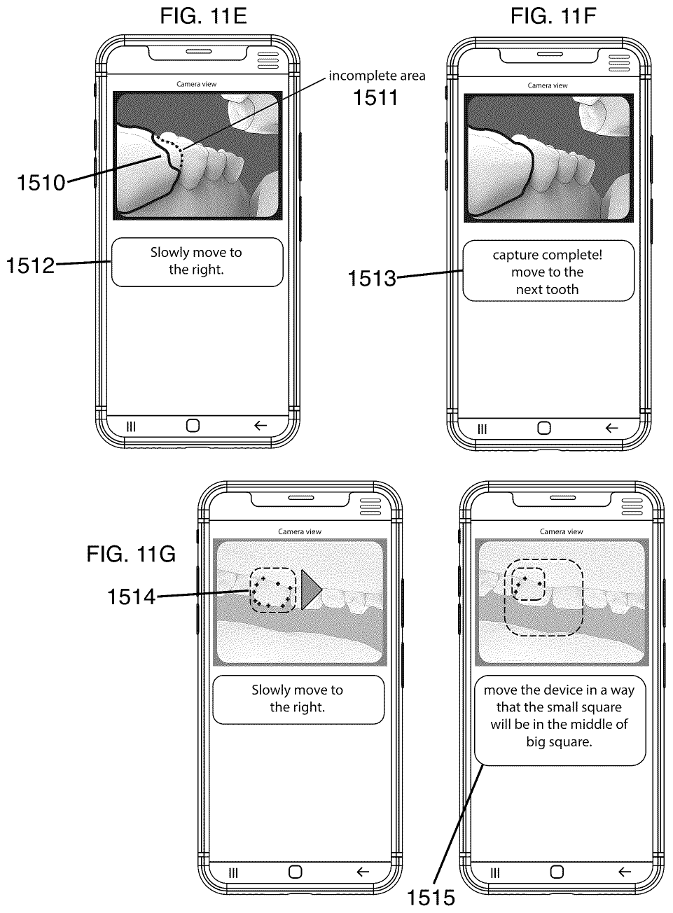

In reconstruction scenarios, such as a wearable headset or a handheld scanner, the motion model does not know the control input. A medical device may be moved faster by one person or than another or jerky movements may occur. As such, constant velocity models may not be effective. However, in the case of a handheld scanner, an application of a computing device may instruct a user in a direction of movement of the handheld scanner. If the user moves too fast or too slow, the application may direct the user to slow down or go faster or to move more smoothly. As such, the user is trained in keeping the handheld scanner at an ideal pace. Although the user may not follow the exact pace when scanning, the directions from the application help the scanning process. During the scanning process, the application may provide the user with guidance. FIG. 11A illustrates an example of a handheld device including a camera 1500. FIG. 11B illustrates an application 1501 of a communication device 1502 displaying a camera view 1503 of the camera 1500 of the handheld device, a suggested direction of movement of the handheld scanner 1504, and a suggested speed of movement 1505 of the handheld scanner. FIG. 11C illustrates the application 1501 displaying the camera view 1503 of the camera 1500 of the handheld device, a guiding message 1506, and an uncertainty level 1507 in that which the handheld scanner is scanning. The uncertainty level 1507 increases with jerky movements. FIG. 11D illustrates the application 1501 displaying the camera view 1503 of the camera 1500 of the handheld device highlighting a particular tooth 1508 and a query 1509 for the user, confirming whether the highlighted tooth is the lower right second premolar as predicted. FIG. 11E illustrates the application 1501 displaying the camera view 1503 of the camera 1500 of the handheld device highlighting a scanned area 1510 of a tooth and an area 1511 in which scanning is incomplete. The application suggests a direction and speed of movement 1512 to complete the scan of the tooth. FIG. 11F illustrates the application 1501 displaying the camera view 1503 of the camera 1500 of the handheld device highlighting the scanned area of the tooth 1510 and a message 1513 indicating the scan of the tooth is complete. FIG. 11G illustrates the application 1501 displaying the camera view 1503 of the camera 1500 of the handheld device during scanning, wherein the tooth of interest is highlighted 1514 and a suggested speed and direction of movement are provided 1515.

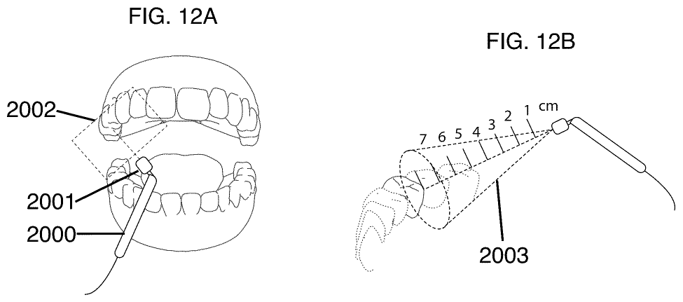

For modeling motion inside a mouth during 3D reconstruction of the teeth, constant velocity is assumed and unexpected accelerations are modeled with a Gaussian profile. FIG. 12A illustrates a handheld device 2000 with a camera 2001. The processor identifies a feature, a tooth, in a first image 2002. FIG. 12B illustrates the handheld device simulating a 3D ray 2003 originating from the camera 2001 of the handheld device 2000. The feature is positioned somewhere along the ray 2003, possible distances from a distribution space shown along a length of the ray 2003. The user is asked, via an application paired with the handheld device 2000, to slightly move the handheld device 2000 in a particular direction. A second image is captured and the ray 2003 is simulated to be superimposed or projected on the second image. Each of the possible distances along the ray 2003 form an ellipse of uncertainty around the feature of interest in the second image. Using Bay's rule for reweighting probabilities of each uncertainty ellipse, and assuming the user follows instructions to move the handheld device 2000 the suggested distance at a constant velocity and acceleration within a range is modeled as a Gaussian, in a few steps the distance converges on one of the possible distances along the ray 2300 with relatively good certainty. In some embodiments, initializing occurs by asking the user, via the application paired with the handheld device, to position the camera of the handheld device at a particular position (e.g., upper right side, facing towards the bottom teeth). After a few iterations, the distance estimate probability density function peaks at a certain possible distance. At next iterations, hypotheses are validated and the process repeats, wherein an image is captured, a distance hypothesis is projected, an uncertainty is created for each possible distance, each of the possible distances with an elliptical are visualized, and the best possible distance is searched for.

In some embodiments, the robot is paired with the application of the communication device by exchanging information between the application and the robot. Information may be transmitted wirelessly using Bluetooth. In some embodiments, user input is provided to at least one of the robot and the application to initiate pairing or progress the pairing process. In some embodiments, pairing the application of the communication device with the robot is initiated by scanning a QR code using a camera of the communication device. Some embodiments may use at least some of the methods, processor, and/or techniques for pairing the robot with the application described in U.S. Non-Provisional patent application Ser. No. 16/109,617, hereby incorporated herein by reference.

In some embodiments, the application displays a map (e.g., of a mouth or an organ) as it is being built and updated. In some embodiments, various map customizations are implemented using the communication device (e.g., mobile phone, tablet, laptop, etc.). For example, a user may use their finger to highlight an area of interest such that the application may provide guidance to the user in guiding a medical device to the area of interest. The application may also be used to label areas within the map (e.g., teeth labels such as molar and bicuspid; organ labels such as kidney and spleen; and other types of labels describing features within the map, such as abnormal or normal, cavity, etc.). In some cases, the application adjusts recommendations to the user, such as which way to move a medical device, based on an updated map. In some cases, the application displays a camera view of a medical device. In some embodiments, the user uses the application to manually control a robot holding a medical device. In some embodiments, a historical report of prior sessions may be accessed by a user using the application. In some embodiments, the historical report may include a total number of operation hours per session, medical notes for each session, the map corresponding to each session, issues encountered in each session, and location of issues encountered (e.g., displayed in the map) in each session.

In some embodiments, the application displays a battery level or charging status and maintenance information of a medical device or robot operating a medical device, firmware information, an icon within the map representing a location of an object. In some embodiments, the amount of time left until full charge or a charge required to complete the remaining of a session may be displayed to the user using the application. In some embodiments, the amount of working time a remaining battery level can provide is displayed by the application. In some embodiments, the amount of time remaining to finish a session is displayed by the application. In some embodiments, the user uses the application to choose a type of task (e.g., type of procedure). In some embodiments, the user may use the user interface of the application to choose preferences. In some embodiments, the user may use the application to view areas at which work is performed on within the map. In some embodiments, the user may use the user interface of the application to add information to the map (e.g., labels, areas of interest, etc.). In some embodiments, the user may use the user interface of the application to create zones within the map. In some embodiments, the application may be used to display a status of the medical device or the robot operating the medical device (e.g., idle, performing work, charging, etc.).

In embodiments, the application of the communication device displays the map in 2D or 3D. In some embodiments, a location of a medical device is shown within the map. In some embodiments, the application may display the map and allow zooming-in or zooming-out of the map. In some embodiments, the user may add flags to the map using the user interface of the application. For example, a flag may be inserted into the map and the flag may indicate a particular diagnosis. In some embodiments, the application may be used to measure a size of an object within the map (e.g., a tooth, an organ, etc.). In some embodiments, flags may be labelled with a name.

In some embodiments, more than one device may be connected to the application and the user may use the application to choose settings for each device. In some embodiments, the user may use the application to display all connected devices. For example, the application may display all devices in a logical representation such as a list with icons and names for each device. In some embodiments, the user may choose which device to use for a session.

In some embodiments, the application may extract a map from a video and/or images captured by a camera of a medical device as the user moves the medical device around its surroundings. In some embodiments, a user may label objects within an image such that the application may identify a location of the objects based on the image data. For example, the user may draw a circle around each object in a video or image and label the object, such as kidney or liver. A text box may pop up and the user may provide a label for the highlighted object. Or in another implementation, a label may be selected from a list of possible labels. Based on the labels provided, the application may associate the objects with respective locations within the map. In some embodiments, a neural network may be trained to recognize different types of objects. In some embodiments, the neural network may be provided with training data and may learn how to recognize different teeth or organs. In some embodiments, a camera captures images or video and using object recognition the application identifies the types of teeth or organs within the images or video captured and associates a location within the map with the object identified. In some embodiments, a robot operating a medical device may be instructed using the application to move the medical device to a particular object and to perform a particular action upon reaching the object (e.g., filling of a bottom right molar tooth).

In some embodiments, input into the application chooses or modifies functions and settings of a medical device or a robot operating a medical device. Settings may include a light intensity of a light source of the medical device, a speed of movement of the medical device, a maximum speed of movement of the medical device, etc.

The map displayed by the application may include several layers. Each layer may include different types of information and the application may be used to turn each layer on or off. Examples of layers include a base layer including an outline of a map, an object layer including object labels, and a medical notes layer including medical notes relating to different objects in the map.

In some embodiments, the application displays the map as its being built in real-time using sensor data captured by one or more sensors of the medical devices as the user or a robot moves the medical device around its surroundings. In some embodiments, the application labels objects within the map and/or creates logical divisions of areas within the map. Separate areas may be displayed in different colors such that may be distinguished from one another. In some embodiments, the application is used change or delete a label. The application may have access to a database of different surroundings (in similar categories such as organs, mouth, etc.) to help in identifying and labeling. In some embodiments, the application displays a scale reference (in feet or meters) within the map such that the user has a rough idea of a size of objects.

In some embodiments, map generation is completed prior to beginning a procedure. In some embodiments, the application displays a request to a user to first generate a map, the user providing an input to the application that confirms or rejects the request. In this mode, the medical device is moved around its surroundings (e.g., mouth, stomach cavity, etc.) to generate the map without performing any other task. In some embodiments, the application determines an estimate of an amount of time required to complete a session, the estimate being displayed by the application.

In some embodiments, the application is configured to receive input designating a working zone or a no-entry zone within the map. In some embodiments, a medical device vibrates when the medical device is close to and/or within a no-entry zone. This feature may prevent users or a robot operating the medical device from operating in sensitive territory during a procedure and ensures work remains within the defined working zone. In some embodiments, the application suggests or autonomously implements working zones and/or no-entry zones.

In embodiments, the medical device and/or the robot operating the medical device is configured to update its understanding of the environment, adjust its settings and/or execute actions according to the at least one input received by the application.

Some embodiments of the application employ a wizard tool to walk the user through different steps of interaction and/or decision making. The wizard provides different options to the user at each step using the application. When a goal of the wizard is to help with decision making the options are presented to the user in a form of questions. The wizard decides between available options based on the user-provided answers to the questions. For example, the wizard helps the user determine the best medical device or procedural settings based on their answers to a series of questions.

In some embodiments, the user uses the application to remotely instruct the robot to capture an image using the camera disposed on the medical device or another camera and the application displays the image. In some embodiments, the user positions the medical device in a desired position and the camera disposed on the medical device captures an image. The images captured may be stored in an internal gallery or album of the application. In addition to the usual metadata, the application tags the images by location within the map (i.e., local/location tagging). The user may access images using a gallery of the application or based on a selected location within the map of which images are tagged. The application may display the map and images captured within different areas within the map. The application may receive user input to export, share, or delete images. If enabled via the application, the robot may send images to the user under certain scenarios. For example, if the robot encounters an unidentified object, the camera captures an image of the object and sends the image to the application. The application may require user input indicating how the robot is to proceed.

Some embodiments may use at least some of the methods, processes, and/or techniques for operating an electronic device (such as the medical device or robot operating the medical device) using an application of a communication device described in U.S. Non-Provisional patent application Ser. Nos. 17/494,251, 17/344,892, 17/670,277, 17/990,743, 15/272,752, 17/878,725, and 18/503,093, each of which is hereby incorporated herein by reference.

In some embodiments, the medical device operated by the user or the robot includes one or more environmental sensors. In some embodiments, a processor of the medical device or the robot adjusts settings of the medical device based on data captured by the environmental sensors. Examples of settings include a light intensity of a light source of the medical device, a speed of movement of the medical device, a maximum speed of movement of the medical device, etc. Examples of sensors include obstacle sensors, acoustic sensors, cameras, optical sensors, distance sensors, motion sensors, tactile sensors, and the like. Sensors may sense various attributes of one or more of these features of an environment, e.g., hardness, location, sliding friction experienced by an instrument, hardness, color, acoustic reflectivity, optical reflectivity, planarity, acoustic response of a surface to an instrument, and the like. In some embodiments, the sensor takes readings of the surroundings (e.g., periodically, like more often than once every 5 seconds, every second, every 500 ms, every 100 ms, or the like or randomly as determined by an algorithm) and the processor obtains the sensor data. In some embodiments, the sensed data is associated with location data of the medical device indicating the location of the medical device at the time the sensor data was obtained. In some embodiments, the processor infers environmental characteristics from the sensory data. In some embodiments, the processor infers characteristics of the environment in real-time from real-time sensory data. In some embodiments, the processor adjusts various operating parameters of actuators, like speed, torque, duty cycle, frequency, flow rate, pressure drop, temperature, brightness, etc. For instance, some embodiments adjust the speed of the medical device (e.g., a scalpel, a scope, a burr, etc.) based on the environmental characteristics inferred (in some cases in real-time according to the preceding sliding windows of time). In some embodiments, the processor activates or deactivates (or modulates intensity of) functions based on the environmental characteristics inferred (a term used broadly and that includes classification and scoring). In other instances, the processor adjusts a cleaning path, operational schedule (e.g., time when various designated areas are worked upon, such as when cleaned), and the like based on sensory data.

In some embodiments, inferred environmental characteristics of different locations of the environment are marked within the map based on observations from all or a portion of current and/or historical sensory data. In some embodiments, the environmental characteristics of different locations within the map are modified as new sensory data is collected and aggregated with sensory data previously collected. In some embodiments, information such as date, time, and location are associated with each sensor reading or other environmental characteristic based thereon. In some embodiments, probabilities of environmental characteristics existing in a particular location of the environment are determined based on current sensor data and/or sensor data collected during prior work sessions. In some embodiments, settings of the medical device or the robot operating the medical device are adjusted based on environmental characteristics with highest probability of existing in the particular location such that they are ideal for the environmental characteristics predicted. In some embodiments, areas with high risk of issues are predicted. In some embodiments, the medical device vibrates when the medical device is close to and/or within a high risk area. This feature may prevent users or a robot operating the medical device from operating in sensitive territory during a procedure. Some embodiments may use machine learning to infer environmental characteristics based on sensor data. Some embodiments may use a classifier such as a convolutional neural network to classify real-time sensor data of a location within the environment into different environmental characteristic classes.

Some embodiments may use at least some of the methods, processes, and/or techniques for autonomously adjusting settings of an electronic device (such as the medical device or robot operating the medical device) described in U.S. Non-Provisional patent application Ser. Nos. 16/239,410, 17/693,946, 17/494,251, 17/344,892, 17/670,277, 17/990,743, and 18/503,093, each of which is hereby incorporated herein by reference.

In some embodiments, the processor uses a neural network to stitch images together and form a map. Various methods may be used independently or in combination in stitching images at overlapping points, such as least square method. Several methods may work in parallel, organized through a neural network to achieve better stitching between images. Particularly with 3D scenarios, using one or more methods in parallel, each method being a neuron working within the bigger network, is advantageous. In embodiments, these methods may be organized in a layered approach. In embodiments, different methods in the network may be activated based on large training sets formulated in advance and on how the information coming into the network (in a specific setting) matches the previous training data.

In some embodiments, a camera, installed on the medical device, for example, measures the depth from the camera to objects within a first field of view. A first segment of the map is constructed from the depth measurements taken within the first field of view. A first recognized area within the working environment is established, bound by the first segment of the map and the outer limits of the first field of view. The camera continuously takes depth measurements to objects within the field of view of the camera. Depth measurements taken within the second field of view are compared to those taken within the first field of view in order to find the overlapping measurements between the two fields of view. Different means for finding overlap may be used. An area of overlap between the two fields of view is identified (e.g., determined) when (e.g., during evaluation a plurality of candidate overlaps) a number of consecutive (e.g., adjacent in pixel space) depths from the first and second fields of view are equal or close in value. Although the value of overlapping depth measurements from the first and second fields of view may not be exactly the same, depths with similar values, to within a tolerance range of one another, can be identified (e.g., determined to correspond based on similarity of the values). Furthermore, identifying matching patterns in the value of depth measurements within the first and second fields of view can also be used in identifying the area of overlap. For example, a sudden increase then decrease in the depth values observed in both sets of measurements may be used to identify the area of overlap. Examples include applying an edge detection algorithm (like Haar or Canny) to the fields of view and aligning edges in the resulting transformed outputs. Other patterns, such as increasing values followed by constant values or constant values followed by decreasing values or any other pattern in the values of the perceived depths, can also be used to estimate the area of overlap. A Jacobian and Hessian matrix can be used to identify such similarities.

In some embodiments, an image sensor, installed on the medical device, acquires data to estimate depths from the image sensor to objects within a first field of view. In some embodiments, the image sensor measures vectors (e.g., two or three dimensional vectors corresponding to physical space) from the image sensor to objects and the L2 norm of the vectors is calculated using ∥x∥_{P}=(Σᵢ|xᵢ|^{P})^{1/P} with P=2 to estimate depths to objects. In some embodiments, each depth estimate is translated into a coordinate by iteratively checking each coordinate within the observed coordinate system of the medical device until the coordinate that coincides with the location of the depth estimated is identified. Each coordinate of the coordinate system coincides with a location within the environment. The coordinate system may be of different types, such as a Cartesian coordinate system, a polar, homogenous, or another type of coordinate system. In some embodiments, coordinates coinciding with the depths estimated are stored in memory in the form of a matrix or finite ordered list.

As the medical device with mounted image sensor translates and rotates within the image sensor continuously acquires data and depths from the image sensor to objects within the field of view of the image sensor are continuously estimated. After estimating depths within each new field of view, depth estimates are translated into coordinates corresponding to the observed coordinate system of the medical device, thereby expanding the discovered areas with each new set of depth estimates.

In some embodiments, map filling and localization begin immediately upon beginning work with the medical device, wherein an initial lack of ground truth is compensated for using probabilistic algorithms.

Embodiments described herein disclose reducing map drift by reducing map filling strength in uncertain circumstances, introducing a non-linear probability map within the map using a sigmoid function, and increasing dynamic range within the map. In some embodiments, reducing map filling strength may be countered by non-linear mapping using the sigmoid function. This stabilizes the map and helps overcome map warping. The conditions and algorithmic improvements disclosed above improve map stability, specifically the execution of immediate mapping. Some embodiments implement a LIDAR scan pitch and roll correction to reduce map warping. LIDAR scan pitch and roll correction may be used to increase scan matching accuracy and consequently map stabilization. Some embodiments implement additional filtering of outliers and discards portions of data that does not fit well with the majority of the data.

In some embodiments, the processor may add different types of information to the map of the environment. In some embodiments, image data information may be inserted at locations within the map from which the image data was captured from. In some embodiments, images may be associated with a location from the images are captured from. In some embodiments, images of areas are stitched together to form a map. In some embodiments, an image may be associated with information such as the location from which the image was captured from, the time and date on which the image was captured, and objects captured within the image. In some embodiments, a user may access the images on the application of the communication device. In some embodiments, the application may sort the images according to a particular filter, such as by date, location, objects within the image, favorites, etc. In some embodiments, the location of different types of objects captured within an image may be recorded or marked with the map.

In some embodiments, the processor post-processes the map before the application displays the map. In some embodiments, the map is processed to clean unwanted areas and noise data.

In some embodiments, multiple maps may be accessed using the application. For example, each map may correspond to a different patient. In some embodiments, a saved map may be loaded and reused for repeat visit or a following procedure by the same patient. The medical device or the robot operating the medical device performs work using the selected map. In some embodiments, a previous environment is autonomously recognized based on sensor data. In some embodiments, a search is performed to compare current sensor observations against data of previously generated maps. In some embodiments, a fit between the current sensor observations and data of a previously generated map may be detected and the saved matching map loaded. However, if immediate detection of the location of the medical device is not possible, a new map is built. As the medical device moves within the environment (e.g., translating and rotating), the likelihood of the search being successful in finding a previous map that fits with the current observations increases as more features that may lead to a successful search are observed. The features observed at a later time may be more pronounced or may be in a brighter environment or may correspond with better examples of the features in the database.

Some embodiments may use at least some of the methods, processes, and/or techniques for creating, updating, and presenting a map of an environment (including information within the map) described in U.S. Non-Provisional patent application Ser. Nos. 16/163,541, 17/494,251, 17/344,892, 17/670,277, 17/990,743, 16/048,185, 16/048,179, 16/920,328, 16/163,562, 16/724,328, 16/163,508, and 18/503,093, each of which is hereby incorporated herein by reference.

In some embodiments, the medical device includes a camera for capturing images from the environment. A structured light source may be disposed on the medical device and emit structured light onto objects within its surroundings, wherein the structured light emitted falls within a field of view of the camera. An object type, an object size, an object position, and/or a depth of an object captured within an image may be determined. Examples of identifiable object types include different organs (e.g., kidney, spleen, gull bladder, liver, etc.), veins or arteries, different teeth (e.g., molar, bicuspid, wisdom tooth, etc.). In some embodiments, images captured are processed locally for privacy. In some embodiments, an object type of an object is added to an object database for use in future classification.

In some embodiments, a light pattern may be emitted onto objects. In some embodiments, time division multiplexing may be used for point generation. In some embodiments, an image sensor may capture images of the light pattern projected onto the object surfaces. In some embodiments, distances to the objects on which the light pattern is projected are inferred based on the distortion, sharpness, and size of light points in the light pattern and the distances between the light points in the light pattern in the captured images. In some embodiments, a distance for each pixel in the captured images is inferred. In some embodiments, a three dimensional image is created based on the inferred distances to objects in the captured images. In some embodiments, a distance to the objects is estimated based on the position of the light in the captured image. In some embodiments, the distance is determined using a table relating position of the light in a captured image to distance to the object on which the light is projected. In some embodiments, using the table comprises finding a match between the observed state and a set of acceptable (or otherwise feasible) values. In embodiments, the size of the projected light on the surface of an object may also change with distance, wherein the projected light may appear smaller when the light source is closer to the object. In some cases, other features may be correlated with distance of the object. The examples provided herein are for the simple case of light project on a flat object surface, however, in reality object surfaces may be more complex and the projected light may scatter differently in response. To solve such complex situations, optimization may be used to provide a value that is most descriptive of the observation. In some embodiments, the optimization may be performed at the sensor level such that processed data is provided to the higher level AI algorithm. In some embodiments, the raw sensor data may be provided to the higher level AI algorithm and the optimization may be performed by the AI algorithm.

In some embodiments, an emitted structured light may have a particular color. In some embodiments, more than one structured light may be emitted. In embodiments, this may improve the accuracy of the predicted feature. For example, a red IR laser or LED and a green IR laser or LED may emit different structured light patterns onto surfaces of objects. The green sensor may not detect (or may less intensely detects) the reflected red light and vice versa. In a captured image of the different projected structured lights, the values of pixels corresponding with illuminated object surfaces may indicate the color of the structured light projected onto the object surfaces. For example, a pixel may have three or four values, such as R (red), G (green), B (blue), and I (intensity), that may indicate to which structured light pattern the pixel corresponds to. Structured light patterns may be the same or different color and may be emitted by the same or different light sources. In some cases, sections of the image may capture different structured light patterns at different times. In some cases, the same light source mechanically or electronically generates different structured light patterns at different time slots. In embodiments, images may be divided into any number of sections. In embodiments, the sections of the images may be various different shapes (e.g., diamond, triangle, rectangle, irregular shape, etc.). In embodiments, the sections of the images may be the same or different shapes.

In some embodiments, a neural network is used to determine a distance of an object based on images of one or more laser beams projected on the object. The neural network may be trained based on training data. Manually predicting all pixel arrangements that are caused by reflection of structured light is difficult and tedious. A lot of manual samples may be gathered and provided to the neural network as training data and the neural network may also learn on its own. To train the neural network, the neural network associates pixel combinations in the captured images with depth readings to the objects on which the beams are reflected in the captured images. The processor trains a neural network by associating pixel combinations in the captured images with depth readings to the objects on which the beams are reflected in the captured images. Many training data points may be gathered, such as millions of data points. After training, the neural network is used to determine a distance of objects based on a position of beams reflected on the objects in a captured image.

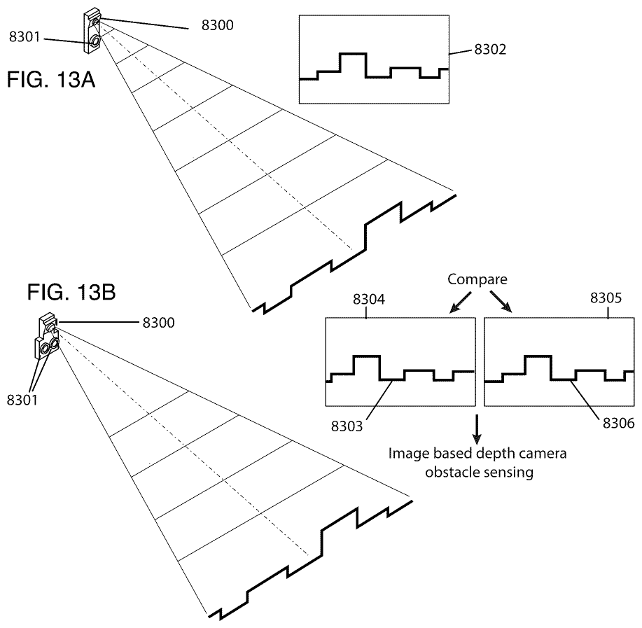

In some embodiments, a combination of a short-range line laser distance measurement device and a depth camera are used to locate near range obstacles. FIGS. 13A and 13B illustrate examples of combinations of line laser 8300 and depth cameras 8301, FIG. 13B including two depth cameras 8301. In FIG. 13A, the line laser 8300 is projected onto obstacles within the environment and the camera 8301 captures an image 8302 of the projected line laser. In FIG. 13B, the projected laser line profile 8303 in image 8304 captured by the cameras 8301 is compared to a saved image 8305 of a laser line profile 8306. Laser line profiles may be unique to a particular object on which the laser line was projected and may therefore be used in identifying the particular object. Laser line profiles may also be unique to a particular location within the environment and may therefore be used in localizing the medical device. A position of the lines forming the laser line profile within captured images may also be used to determine a distance of the object on which the laser line was projected. For example, for the line lasers 8300 angled downward, lines of the laser line profile 8303 positioned lower relative to a top edge of the image 8304 are further away. A position of a line may be correlated with a true distance to the object one which the line is projected. In embodiments, the combined line laser and depth camera are disposed on the medical device.

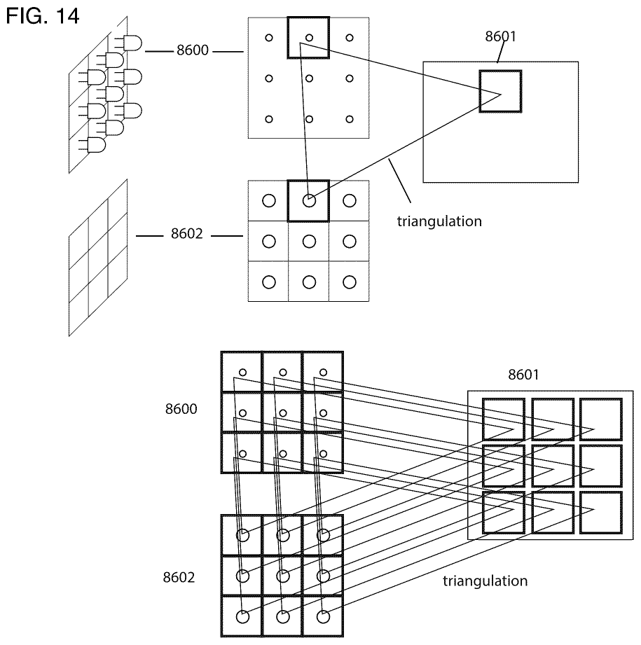

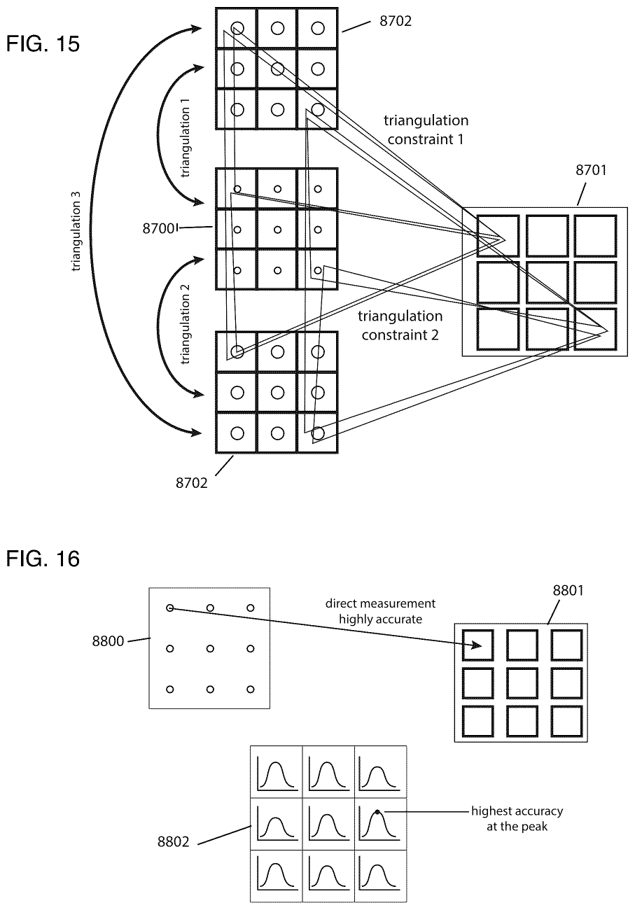

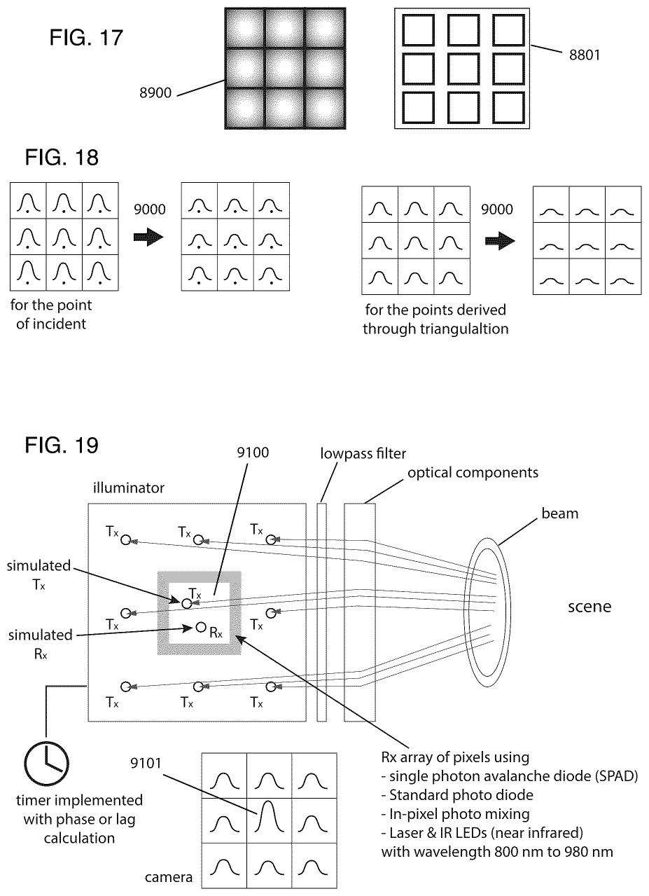

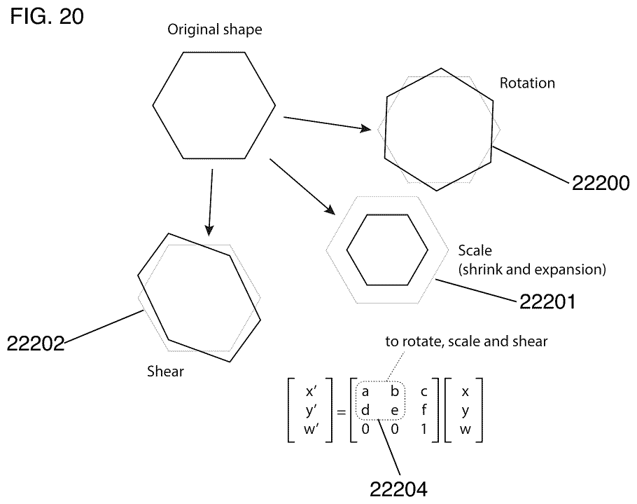

In some embodiments, triangulation is used. FIG. 14 illustrates an example of triangulation between a structured illumination 8600, a scene 8601, and an image 8602 of the scene 8601. FIG. 15 illustrates an example of three different triangulations between a structured illumination 8700, a scene 8701, and two images 8702 of the scene 8701, triangulation one and two each having their own constraint. In some cases, structured light illumination may be used with TOF sensors, such as ST micro FlightSense. A TOF sensor provides an accurate (i.e., occlusion free) reading from a single point that has an incident with the surroundings whereas triangulation provides less accurate data for a larger area of the scene. FIG. 16 illustrates structured illumination using structured TOF sensors 8800 instead of structured light, each TOF sensor directly measuring a distance from a scene 8801 with high accuracy. For each reading of the final readings 8802, the highest accuracy is at the peak. FIG. 17 illustrates the accuracy of each distance measurement point in an image 8900 of the scene 8801 with color intensity. After a time step, the accuracy of measured distances decreases. FIG. 18 illustrates a in accuracy after a time step 9000 for TOF sensor measurement points and for measurements derived through triangulation. The decrease in accuracy is illustrated by a reduction in a height of the peaks and increase in a wideness of the peaks. In some embodiments, point measurements use direct pulse modulation. Pulsed light provides a high energy pulse in a short time, permitting light signals to noise ratio, which performs well under ambient light. The light receiver or a stop-watch sets a start of a counter when a pulse is transmitted from an LED or laser diode while waiting for a return signal. The return signal is often heavily attenuated due to surrounding conditions and the nature and material of objects from which the light is reflected. In embodiments, it is unessential for an emitter and a receiver to be a pair in a structured TOF illumination. For example, a single transmitter may be received by multiple receivers. FIG. 19 illustrates a relation between confidence score and a location of transmitters Tx and receiver Rx in an illuminator positioned behind a lowpass filter and optical components and illuminating the scene. Since a transmitter and receiver are only co-located in location 9100, the confidence score of measurement 9101 is increased as the position corresponds to the location 9100. The remaining confidence scores are decreased as they are positioned a distance from the receiver. In some embodiments, continuous wave modulation may use homodyne amplitude modulation to modulate a sinusoidal or square wave.

In some embodiments, a number of points of reflection are correlated with distance or angular resolution of a depth camera. The points of reflection may comprise a number of points whose connections form a map of the surroundings. In some embodiments, a mathematical formula or probabilistic method is used to connect the points. As the medical device moves, noise is introduced and some of the points appear off in position after adjusting for the motion of the medical device. Therefore, a probabilistic method may be used to connect the points in a way that has a highest chance of being a reflection of the environment. When the medical device is displaced or a location of the medical device is lost in the map current surrounding points may be searched against one or more previously known maps to rediscover the location of the medical device.

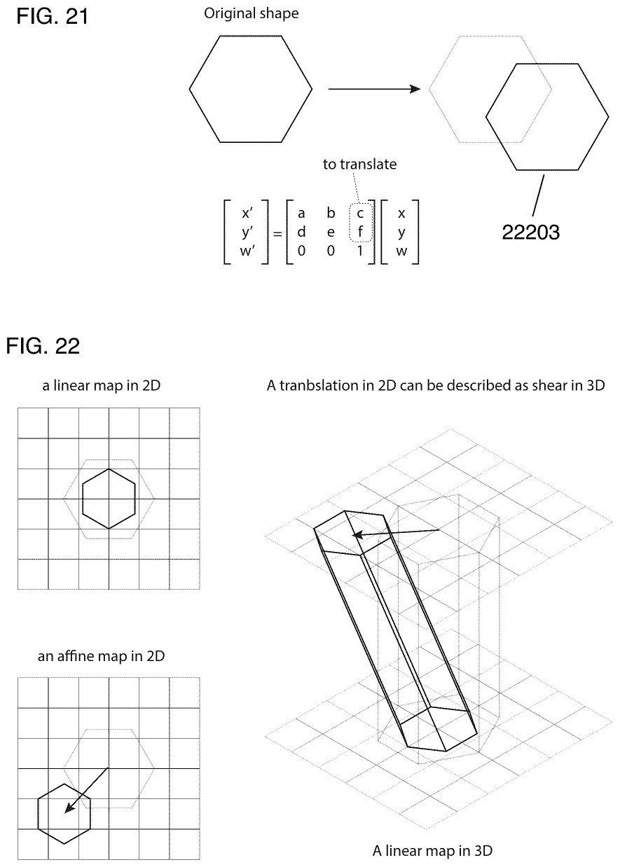

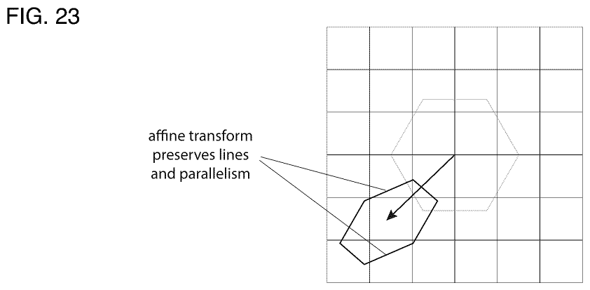

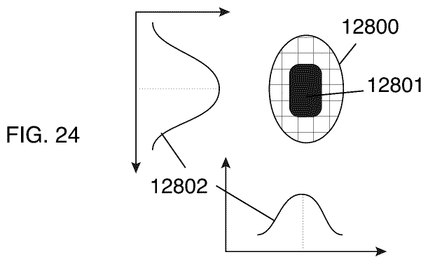

Similar to fine-tuning depth extraction with a sequence of captured images, object recognition may be enhanced using a sequence of images and depth information. Depth information may be extracted from a sequence of images passively from which a point cloud is formed. A sequence of point clouds arranged in a data structure like an image forms a data cube. The point cloud and depth information do not need to be extracted from passive images. Active illumination, whether in an optical method (such as structured light) or in a TOF method, may separately add accuracy, density, and dimension to the passive method. A temporal tuning of a depth extraction or object recognition over a sequence of readings captured from a single point of view or from a moving source (such as the robot equipped with an encoder, gyroscope, optical tracking sensor, IMU, etc.) increases density and improves the likelihood of a better depth extraction or object recognition. A depth may be associated with a recognized feature in an image or a point cloud may be featurized by overlaying two readings. In some embodiments, a biholomorphic map is written as a transformation w=f(z) which preserves angles. Conversely, an isogonal map preserves the magnitude of angles but not the orientation. When the condition of orientation preservation of local angles is met, conformal mapping or a biholomorphic map is achieved. A similarity transformation is a conformal mapping that transforms objects in space to similar objects, wherein mathematically A and A′ are similar matrices and A′=BAB⁻¹. A similarity transformation allows uniform scaling with at least one more degree of freedom than Euclidean transformation. The transformation is an affine transformation when the condition of preserving collinearity is met, wherein all points on a line still form a line after an affine transform and ratios of distances remain preserved. Affine transformation may be decomposed to rotations, translation, dilations, and shears. FIGS. 20 and 21 illustrate affine transformations decomposed to rotation 22200, scale 22201, shear 22202, and translation 22203 and the variable in the matrix 22204 applying the transformation. FIG. 22 illustrates a translation in 2D described as a shear in 3D. FIG. 23 illustrates preservations of lines and parallelism in affine transformation. FIG. 390 illustrates translation relating different perspectives within the affine space.

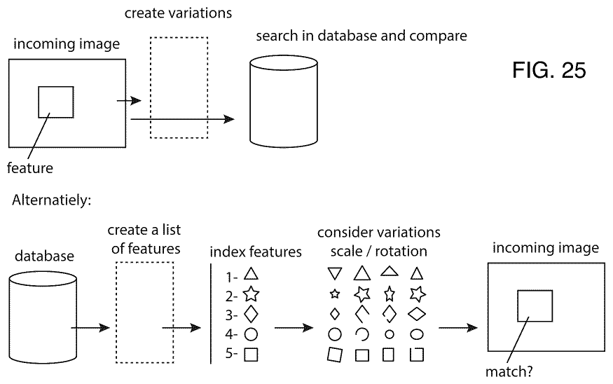

In some embodiments, a bounding volume is used to visualize an object. FIG. 24 illustrates a bounding volume 12800 surrounding object 12801 with an uncertainty cloud including mean and variance 12802. The bounding volume inflates or deflates depending on a confidence score of the identified object 12800. In embodiments, inflation or deflation is determined using naive methods or MDP (i.e., reinforcement learning).

In some embodiments, the input data of a neural network comprises spatial correlation with objects in a surrounding environment. In some embodiments, each dimension in a layer is associated with a dimension in the input. In some embodiments, the input data comprises an image or a stream of images. In some embodiments, width and height dimensions may correlate to 2D features of an image. In some embodiments, width, height, and depth dimensions may correlate to 2D features and depth of a depth image. In some embodiments, an area of an image comprising an object of interest is identified and analyzed to determine a likelihood of a presence of the object based on probability scores of the object belonging to various categories or classes of objects. In simulation tools, a boundary box may be used to determine a perimeter of a detected object. In some embodiments, the system is designed to reduce computational cost of detection of objects with no loss of accuracy. In some embodiments, an early determination is made as to whether processing a portion of a high-resolution image is likely to return a spent value.

In some embodiments, network output is based on training and is not hard coded by a human written algorithm. In some embodiments, training is executed on an external hardware. In some embodiments, design of a network layer and their logical operations does not necessitate a separation of hardware or software along logical separations. In some embodiments, training input data comprises examples labeled by a human or an algorithm that automatically generates labeled data or both. In some embodiments, training input data comprises 2D images captured by a camera or depth information captured by a depth camera. In some embodiments, classification may be executed for 2D images captured by a camera or for 3D or 2D depth data captured by a depth camera. In some embodiments, classification may be executed for images captured by a camera or for 3D or 2D depth data captured by a depth camera, separately or in combination.

In some embodiments, initial processing uses lower resolution to quickly determine if further high-resolution processing is required. This decision may be made in real-time. In some embodiments, upon determining a probability exceeding a threshold that an object of interest is identified in the input data, further processing is executed to return a more accurate prediction. In some embodiments, after initial processing, unnecessary input data is pruned and is not further processed to provide faster processing of select input data. Processing of the select data may occur in real-time. In some embodiments, low accuracy processing requiring lower computational budget is carried out on a wider group of inputs while high accuracy processing requiring higher computational budget is carried out on a select group of inputs. In some embodiments, separation of inputs into high and low computational budgets permits real-time processing of both. In some embodiments, training input data comprises an image with a label of an object within the image, a label of an object within the image and a bounding box defining an area of the image within which the object appears, multiple labels of multiple objects within the image, multiple labels of multiple objects each with a bounding box, or a selection of labels of objects within a bounding box. In some embodiments, training input data comprises multiple channels, one for each color and/or one for grayscale color. In some embodiments, training the network using labeled examples occurs over a series of runs, wherein during each run at least backpropagation is used to decide proper values for parameters. In some embodiments, the final values for parameters are used as a reference model to predict labels and bounding boxes for objects detected in new input data.

In some embodiments, regions in an image corresponds to regions in a spatial proximity of the medical device. In some embodiments, a 2D image is illuminated using one of a light, an IR sensor, and a laser, to provide depth information. In some embodiments, the illumination may occur during alternating time slots and/or for alternating images. In some embodiments, a 2D image may include multiple channels, such as R, G, B, depth, and grayscale. In some embodiments, a machine learning network is applied to each of the channels individually, acting as a subsystem, the results of which are combined at the end. In some embodiments, the network may be applied to a combination of channels. In some embodiments, the network may be applied to a group of RGB or greyscale channels as a subsystem and separately applied to depth, the results of which are combined in weighted manner. In some embodiments, illumination on a 2D image may be achieved by a depth camera that measures depth. In some embodiments, the depth camera provides depth information that is fed into a separate network of computational nodes independent of a network that receives 2D images as input. In some embodiments, the results obtained from each of the networks independently provides probabilities of existence of an object within a near field or far field vicinity of the medical device.

In some embodiments, a first subsystem network validates a result of a second subsystem network to provide a highly reliable system. In some embodiments, a LIDAR projects active illumination, a reflection of which is received by its receiver for depth measurement. In some embodiments, the illumination is simultaneously captured by a 2D camera, the illumination falling within a FOV of the camera and the receiver of the LIDAR. In some embodiments, a neural network of nodes generates a probability score indicating a likelihood that a region in an image includes an object belonging to a category or class of object present in the surroundings of the medical device. In some embodiments, objects detected in the surroundings of the medical device are stationary or dynamic in nature. In some embodiments, possible trajectories, speed, and direction of movement of dynamic objects are scored to predict their location in a next time slot. In some embodiments, probability scores are determined based on training examples rather than pre-coded algorithms. In some embodiments, probability scores obtained based on training examples are used for formulating movement decisions of the medical device. In some embodiments, probability scores and corresponding categorization are transmitted to a remote control center where human assistance is provided to help in making more complicated decisions. In some embodiments, probability scores may be communicated to a user via a light, sound, LCD display, or another method. In some embodiments, active illumination sensing is comprised of measuring any of a distance, a turnaround time, a direction, an intensity, and a phase shift reflected from a surface on which an illumination signal is projected.

Concurrently, as the map us built, the medical device is localized, the object of interest solidifies, and more characteristics of the object are discovered. Examples of characteristics include size and a distance of the object. As more areas are observed, the object becomes better defined, wherein probabilities of its size, distance, and nature (i.e., an object type of the object) converge to more solid estimations (i.e., estimations with greater confidence). At some point, the object of interest is classified with reasonable accuracy. Similarly, as sensors scan more areas of the environment, more boundaries and objects are discovered.

Upon completion of a map, objects discovered including their proposed labeling are presented to a user using the application of the communication device. The user uses the application to accept or reject the proposed labeling by, for example, swiping right or left or using another gesture on the screen of the communication device or by providing another form of user input. This helps train the algorithm to properly label objects. Some embodiments use training methods. In one example, the application proposes labeling a discovered object as a liver. Upon swiping right to indicate a correct classification, the label-object pair is provided as input to the ML or DNN algorithm and the reinforcement is used in improving future classifications. However, upon swiping left to indicate an incorrect classification, the label is removed from the object and is recognized as a misclassification. The information is provided as input to the ML or DNN algorithm to improve future classifications. In some embodiments, another label for the object is proposed to the user using the application based on a next best prediction of an object type of the object.

A learning mechanism may be comprised of two separate subsystems. One subsystem of the learning mechanism is global and the global training subsystem comprises a large data set of labeled images, wherein the images comprise images labelled by staffed human operators and/or crowd sourced images labelled by users and/or auto-generated labelled images, and/or a combination of the above. The other subsystem of the learning mechanism is local, wherein the local training subsystem includes fewer objects and classes that are typically found in a specific environment with more granular labelling. In some embodiments, the global training and object classification subsystem is used as an a priori to narrow down an object type of an object, while the local training and object classification subsystem provides further fine tuning in determining the object type. In some embodiments, an unsupervised method is locally used to cluster objects into categories, then a supervised method is applied only a subset of the data (e.g., each category or the images of that category).

In some embodiments, an object is classified or the object is kept unclassified based on the consequences defined for a wrong classification. For instance, more conservative classification may be used for objects when a wrong classification results in an assigned punishment, such as a negative reward. In contrast, more liberal classification of objects may be used when there are no consequences of misclassification of an object. In some embodiments, different objects may have different consequences for misclassification of the object. For example, a large negative reward may be assigned for misclassifying an object as a tumor. In some embodiments, the consequences of misclassification of an object depends on the type of the object and the likelihood of encountering the particular type of object. In some embodiments, the likelihood of encountering a particular type of object is determined based on a collection of past experiences with patients.

Some embodiments are initially be trained in classification of objects based on a collection of past experiences. Some embodiments are further trained in object classification using user feedback. In some embodiments, the user may review object classifications using the application of the communication device and confirm the classification as correct or reclassify an object misclassified. In some embodiments, the processor adjusts the weight given to classification based on the collection of past experiences and user feedback. In some embodiments, the weight is preconfigured. In some embodiments, the weight is adjusted by a user using the application of the communication device paired.

In some embodiments, the processor classifies the type, size, texture, and nature of objects. Some embodiments determine a generalization of an object based on its characteristics and features. Generalization of objects may vary depending on the characteristics and features considered in forming the generalization. Due to the curse of dimensionality, there is a limit to the number of characteristics and features that may be used in generalizing an object. Therefore, a set of best features that best represents an object is used in generalizing the object. In embodiments, different objects have differing best features that best represent them. In some embodiments, determining the best features that best represent an object requires considering the goal of identifying the object; defining the object; and determining which features best represent the object. In some embodiments, determining the best features that best represents an object and the answers to such considerations depends on the actuation decision of the medical device upon encountering the object.

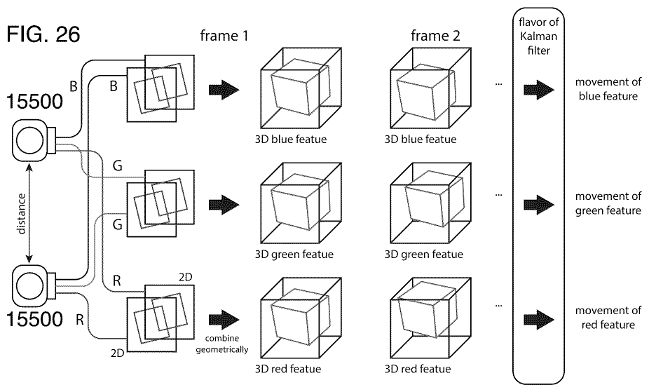

In some embodiments, images are reduced to features. For example, FIG. 25 illustrates the processor extracting a feature from an incoming image 1800 and performing a search through previously captured images in a database 1801 to find a match to previously observed features captured in the previous images. In some embodiments, the opposite is implemented. For example, FIG. 25 also illustrates creating and indexing features captured in previous images and their variations 1802, and searching an incoming image 1803 to determine if it includes any of the features observed previously in the previous images. A frontend algorithm or a backend algorithm may be used to search for a match between features. In a frontend algorithm, a matching test between compared features may be simplified to a binary test, may use reduced resolution, and/or may reduce the features to be tested to only a few strongest features or a strongest feature. A backend algorithm is only useful when performed online. As such, a backend algorithm may concurrently begin a more investigative approach as a backup to the frontend algorithm. If the frontend algorithm fails to find a match, the backend algorithm may find a match, although may take more time in finding the match.

In some embodiments, the user demonstrates an action the robot operating the medical device is to perform upon identifying a particular object. In some embodiments, the user demonstrates an action the robot is to perform upon identifying a particular object using virtual reality.

Some embodiments comprise any of semantic segmentation, object extraction, object labeling, object depth assignment, bounding box depth assignment, tracking of bounding box, continuous depth calculation of bounding box of an object of interest. Properties that may be associated with the object include dimensions and size of the object; surface characteristics of the object (e.g., level of reflectiveness, color, roundness, smoothness, texture, roughness); corners, edges, lines, or blobs of the object; a direction of movement of the object, including absolute movement and relative movement; a direction of acceleration or deceleration; static or dynamic property of the object; sensors from which the object is hidden; occlusion, partial occlusion, previous occlusion, or approaching occlusion of the object; and a level of influence of environmental factors on the object (e.g., lighting conditions). Some properties associated with the object depend on other observations. For example, absolute depth in relation to a frame of reference depends on processing/rendering of at least a portion of a map. Additionally, there is partial observability while data is gathered for processing/rendering the map, and while there is observance of some values of properties, a lower confidence level is assigned to those values. Probabilistic values or descriptions of one or more properties associated with an object depends on sample data collected at a current time and up to the current time. In cases wherein partial observable data is used, principals of central limit theorem are used, assuming a mean of a large sample population is normally distributed and approaches a mean of the population and a variance of the sample population approaches a variance of the original population divided by a size of the sample.

In some embodiments, objects are detected using a short range structured light and camera pair. In some embodiments, obstacles are detected using stereo matching on two cameras. In some embodiments, at least one camera is used for object detection or finding a volume of an object. In some embodiments, two or more cameras are used. In some embodiments, patches of a first image and patches of a second image captured by a first camera and a second camera, respectively, are matched by a sum of absolute differences, a sum of squared differences, cross correlation, census transform and similar methods, bundle adjustment, etc. In some embodiments, the Levenberg Marquardt algorithm is used for optimization. In some embodiments, corners of an object are used as the patch using SIFT. In some embodiments, Harris, Shi Tomasi, SUSAN, MSER, HOG, FAST or other methods are used for detecting and matching patches of images. In some embodiments, SURF and other methods are used to identify a desired patch among multiple patches in each of the images. In some embodiments, features or identified patches are tracked over multiple time steps. In some embodiments, decomposition methods are used to separate localization from feature tracking. In some embodiments, the Lukas-Kanade method is used to assume a constant optical flow in tracking features or patches over time steps. In some embodiments, median filtering or other methods of filtering are used. In some embodiments, convolutional neural networks are used to solve the SLAM problem.

In embodiments, processing of an image or point cloud includes, but is not limited to, any of object noise reduction, object classification, object identification, object verification, object detection, object feature detection, object recognition, object confirmation, object separation and object depth determination. Such image and/or point cloud processing is used to extract meaningful evidence from noisy sensors to, for example, determine a category of a sensed object, an object type, which features from sensed data determine existence of objects within the environment, how an object is separated from other spatially sensed data and which borders form the separation, depth of an object, direction of movement of an object, and acceleration or speed of an object.

Some embodiments may use at least some of the methods, processes, and/or techniques for classifying objects and identifying an object type of objects described in U.S. Non-Provisional patent application Ser. Nos. 17/494,251, 17/344,892, 17/670,277, 17/990,743, 15/976,853, 15/442,992, 16/832,180, 17/403,292, 16/995,500, and 18/503,093, each of which is hereby incorporated herein by reference.

Some embodiments may use at least some of the methods, processes, and/or techniques for determining a distance of an object from the robot or a sensor thereof described in U.S. Non-Provisional patent application Ser. Nos. 17/494,251, 17/344,892, 17/670,277, 17/990,743, 15/447,122, 16/932,495, 15/257,798, 15/243,783, 15/954,410, 16/832,221, 15/224,442, 15/674,310, 15/683,255, and 18/503,093, each of which is hereby incorporated herein by reference.

Some embodiments employ semantic object-based localization. A subtle distinction exists between object associated localization and spatial localization, whether traditional, contextual (semantic), or when combined with object recognition. Object associated localization dictates a behavior (e.g., of a robot operating the medical device) relating to the object and the behaviour is maintained despite the object being located in different locations. For example, object associated localization may be used to cause the robot to perform a same type of action whenever a particular object type is observed. For instance, whenever plaque is observed the robot operates a scraper to remove the plaque. Object associated localization is distinct and different from annotating coordinates corresponding to locations of detected objects. In some embodiments, a behavior or an action is attached to a coordinate system of an object rather than a coordinate system of the environment.

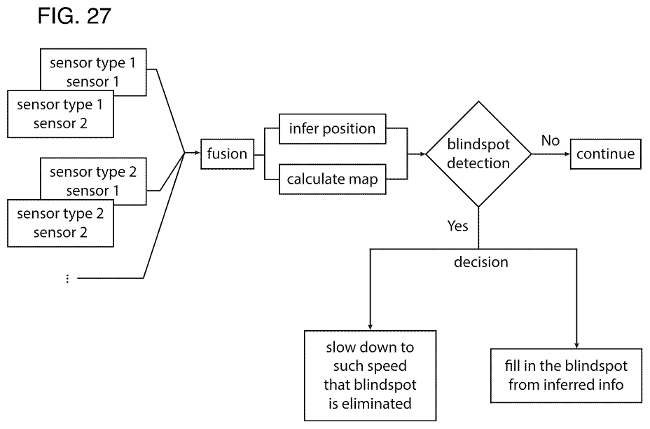

In some embodiments, each data channel is processed for a different clue or feature during image analysis. For example, data output from red, green, and blue (R, G, B) channels are each processed for a specific feature. The green channel is processed for feature 1, wherein feature 1 is detected and tracked from frame to frame using a tracking method, such as Lukas Kanade. In a different channel, such as the red channel, a different feature, feature 2, is dominant and tracked from frame to frame. As such, multiple features are tracked, wherein each channel tracks only one feature. In some embodiments, multiple cameras capture data and corresponding channels are combined based on a geometric association between the cameras defined by a base distance and angle of FOV of the cameras. FIG. 26 illustrates two cameras 15500 positioned a distance from each other. Each camera outputs R, G, and B channels and corresponding channels from each of the cameras 15500 are combined based on a geometric association between the cameras 15500, in this case the distance between the cameras 15500. Each channel tracks a different feature from frame to frame and using a Kalman filter, wherein movement of the tracked features is provided as output.

Some embodiments apply segmentation of foreground and background. For example, an image sensor captures an image. Pixel similarities and spatial proximity are used to separate the image into different segments. Some embodiments partition size of groups with similarity and proximity to cut costs using methods such as K-means clustering, Chan-Vese model energy, wherein a collection of closed curves separates the image into regions, and other clustering methods.

Some embodiments use a graph cut to segment an image. A graph cut splits a directed graph into two or more disconnected graphs. Using a graph cut an image is segmented into two or more regions, wherein similar pixels in close proximity to each other remain in a same segment. In embodiments, cost of a graph cut is determined as a sum of edge weights of the cuts, wherein the cut comprises a subset of all edges. In embodiments, the edges selected are within the cut set such that the sum of the edge weights is minimized. In some embodiments, a source node and sink node (i.e., vertex) are used and only a subset of edges separating the source node and sink node are viable options to form a cut set. Some embodiments employ maximum flow and minimum cut theorem, wherein finding a minimum weight cut is equivalent to finding the maximum flow running between the sink node and the source node. Some embodiments select a sink node such that every pixel node has an outgoing edge to the sink node and select a source node such that every pixel node has an incoming edge from the source node, wherein every pixel node has one incoming edge and one outgoing edge to each of its neighbor pixels. Each pixel connects to the foreground and background (i.e., the source and the sink, respectively), with weights having an equal probability. In some embodiments, pixel similarities are weighted and an algorithm executed by the processor decides a contour of a segment cut. In some embodiments, an online version of a segment cut is combined with a previously trained algorithm.

Segmentation of foreground and background is easiest when the medical device is stationary, however, motion blur occurs as moving objects in the scene cause fluctuating pixel values which influence the spatial and proximity methods described. Some embodiments employ motion compensation using a range of methods, such as phase image differences when the medical device moves linearly in relation to the environment or the environment moves linearly in relation to the stationary medical device. In cases where the medical device moves with constant speed and some objects within the environment move, sensors observe linear change in relation to the fixed environment but not in relation to the moving objects. Some embodiments employ opposite FOV optical flow analysis to identify and distinguish moving objects from the stationary environment, however, the blur from motion still remains a challenge with the above-described methods. In some embodiments, as a complementary measure, a TOF camera captures distances to objects. Although distance measurements also blur as a result of motion, the additional information contributes to an increasingly crisp separation. In addition, in some embodiments, an illumination light with a modulation frequency emitted and phase shift of the returning signal is measured. When an incoherent IR light is emitted, the frequency is changed at different time stamps and each frequency is compared with the frequency of the respective returned IR light.

While the blur effect worsens at higher medical device and/or object speeds, in some embodiments, knowledge of movement of the medical device via sensor data helps transpose the pixel values as a function of time, a function of measured motion, or a weighted combination of both functions. In embodiments, a transpose function of raw pixel values of pixels is defined to shift the pixel values linearly and according to a motion model, which is verified with new sensor data input at a next time step. In some embodiments, there is more than one candidate transpose function. In some embodiments, sum of squared differences is used to select the best transpose function from all candidate transpose functions. A transpose function may include a linear component and an angular component. In some embodiments, optical flow is used to estimate the transpose function when the robot is vision-based. During movement, the medical device may not end at a location intended, however, control commands of a control system may be used to predict a range of possible transpose functions, thereby reducing the search space. In some embodiments, various methods and techniques may be combined, such as multiple phase shift imaging and phase unwrapping methods, or a Fourier transform may be used to model the phase nature of such methods.

In the simplest embodiment, a pixel is assigned a probability of belonging to foreground, background, or unclassified. In a more sophisticated embodiment, multiple depth intervals are defined. Each interval is related to the resolution of the distance measurement. The algorithm sweeps through each pixel of the image, determines an associated probability for each pixel, and performs a classification. For better performance and less computational burden, some pixels may be grouped together and analyzed in a batch. In some embodiments, the probability is determined based on at least neighboring pixel values and probabilities and/or distance measurements associated with pixels. In some embodiments, only pixels of importance are examined. For example, pixels subject to motion blur need resolution as to whether they belong to foreground or background. In some embodiments, depth values known for some pixels are extrapolated to other pixels based on color segmentation, contour segmentation, and edge finding. In some embodiments, a depth sensor associates four data elements with each pixel, depth and R, G, B.

As the medical device moves, motion blur occurs and the blurry pixels require identification as foreground or background in relation to pixels surrounding them. Blurry pixels need to be resolved into one or the other group of pixels. Before or after such resolution occurs, each pixel may have a probabilistic confidence score associated with it. In some embodiments, each pixel is a node in a graph, wherein the edges provide a relation between the connected nodes. In some embodiments, a cost function relates depth values read with color values read. Alternatively, depth values are compared against a combined metric derived from R, G, B, such as grayscale or a more complex metric. Motion blur occurs due to movement of the medical device and movement of objects. In some embodiments, Gaussian Mixture Model, Bayesian methods, and/or statistical weight assignment, are used in one, two or all four data channels (i.e., depth, R, G, B) for improved segmentation. In some embodiments, an HD map, prior training phase map, and/or a vector field map are created with readings captured during an absence of dynamic objects, readings captured with a slow-moving medical device, or readings captured with highly accurate sensors.

Some embodiments follow a contour of colors and shapes. Some embodiments separate closed contours from open contours. Some embodiments separate internal contours from external contours. Some embodiments compare a dominance of all closed shapes in terms of vividness, size, and distinct borders, assign a weight to the closed shapes, normalize, and select one closed shape to follow or track. Some embodiments select one or more closed shapes as feasible successors. Some embodiments track feasible successors with lower resolution to reduce computational intensity. For computational purposes, simple geometric shapes may be preferred, such as a square, a blob, a circle, an oval, a rectangle, and a triangle. In some embodiments, a tracking function inspects the contour being followed at intervals to ensure it is not another similar contour belonging to a different object.

Some embodiments use polyadic arrangement of layers, synaptic connection, and homodyne AM modulation. Some embodiments use semantic segmentation, wherein each segment is associated to a specific category of objects. Some embodiments use semantic depth segmentation, wherein each segment is associated to a depth value. Each type of segmentation may be used to enhance the other or used separately for different purposes.

Some embodiments use a depth image as a stencil to shape or mold a color image. A depth image may be visualized as a grayscale image, has more clear-cut boundaries, and is unaffected by texture and pattern. Challenges remain in touching objects or depth camouflage. In embodiments, a pixel of a 2D image is associated with R, G, B color values that may be used as an input for various processing algorithms. In embodiments considering depth (d), a six-dimensional data structure is formed, wherein instead of associating R, G, B values to an i, j pixel, R, G, B values are associated with an i, j, and d pixel. When such data is used in creating a map of the environment, an outside frame of reference is used. Therefore, depth is translated into a 3D coordinate system of the environment within which the medical device is moving. R, G, B values are associated with the translated depth coordinate in the frame of reference of the environment. This is equivalent to creating a colored map from a colored point cloud.

Clustering may be performed on a pixel for depth, color, or grayscale. Examples of clustering methods include K-means, mean-shift, and spectral clustering. Derivatives and gradient, intensity, and amplitude of depth measurement may be used in understanding how objects in a scene are related or unrelated. In some embodiments, features are extracted using SIFT, HOG, CANNY, etc. methods and are crafted manually. In some embodiments, a raw image is provided as input to a deep network for pre-processing before classification of pixels. Classification methods such as Randomized Decision Forest, SVM, Conditional Random Field, etc. may be used to categorize pixels into depth categories/depth segmentations, wherein there is a depth value for each pixel and/or an object label for each pixel. In addition to pixels having depth and object labels, other labels, such as dynamic or stationary labels, approaching or moving away labels, speed of approach labels, direction of approach labels may be defined.

Some embodiments use at least some methods, processes, and/or techniques for image analysis described in U.S. Non-Provisional patent application Ser. Nos. 17/494,251, 17/344,892, 17/670,277, 17/990,743, and 18/503,093, each of which is hereby incorporated herein by reference.

Some embodiments implement Markov localization. Markov localization uses a probabilistic framework, wherein a probability density over all possible medical device positions in a position space is maintained throughout a session. Initially, the position of the medical device is represented by a uniform distribution, given the location of the medical device is unknown, or a Gaussian distribution, given the location of the medical device is known with high certainty. When the sensor data lacks landmarks, localization is prone to failure. In some embodiments, the iterative nature of optimization is encoded as cycles that evolve with time, like a wave function that is carried forward in a phase space, inspired by Schrodinger's equation of motion. Carrying forward a wave function with time allows for sensor information to be used as its received, with intermittent and dynamic loop closure or bundle adjustment. An adjustment may be seen as a collapse of a wave function upon observation.

In some embodiments, global localization techniques use Markov localization. Markov localization is iterative in nature and may be modeled or encoded in a cyclic data structure. In some embodiments, a Fourier transform is used in conjunction with a probabilistic framework, wherein a probability density over all possible medical device positions in a position space is maintained. Initially, the position of the medical device is represented by a uniform distribution if the location of the medical device is unknown or by Gaussian distribution at times when the location of the medical device is known with a degree of certainty. A cyclic counter may determine a degree of confidence as it increases or decreases with time. At startup, the algorithm starts building a new map and frequently attempts to match the new map with a saved map loaded from storage. When data does not contain any landmarks, the algorithm concludes that it is in a new environment and at intervals checks for known landmarks such that the algorithm knows the medical device has entered a previously known environment and stitches the new temporary map to a previous map to which a match was found based on overlapping points or features. The algorithm may attempt to find a match a predetermined number of times or at intervals. The interval may be longer as time passes. A frontier exploration may be used to help find a match if the algorithm fails to find a match initially. When a match is found, the new map is merged and be superimposed with the previous persistent map with which a match was found. The algorithm may successfully merge the new map with the persistent map, relocalize the medical device, and continue to a next state. If the algorithm is unsuccessful in finding a match, the algorithm continues to build the new map.

In some embodiments, the position of the medical device is tracked as the medical device moves from a known state to a next discrete state. The next discrete state may be a state within one or more layers of superimposed Cartesian (or other type) coordinate system, wherein some ordered pairs may be marked as possible obstacles. In some embodiments, an inverse measurement model may be used when filling obstacle data into the coordinate system to indicate object occupancy, free space, or probability of object occupancy. In some embodiments, an uncertainty of the pose of the medical device and the state space surrounding the medical device are determined. Some embodiments may use a Markov assumption, wherein each state is a complete summary of the past and used to determine the next state of the medical device. In some embodiments, a probability distribution is used to estimate a state of the medical device. In some embodiments, the probability distribution may be determined based on readings collected by sensors. In some embodiments, an Extended Kalman Filter is used for non-linear problems. In some embodiments, an ensemble consisting of a large number of virtual copies of the medical device are used, each virtual copy representing a possible state that the real medical device is in. Embodiments may maintain, increase, or decrease the size of the ensemble as needed. Embodiments may renew, weaken, or strengthen the virtual copy members of the ensemble. In some embodiments, a most feasible member and one or more feasible successors of the most feasible member is determined. Some embodiments use maximum likelihood methods to determine the most likely member to correspond with the real medical device at each point in time. Some embodiments determine and adjust the ensemble based on sensor readings.

Some embodiments use a non-parametric method wherein an ensemble of simulated medical devices and objects is generated, each simulation having different relative position between the simulated medical device and object, the majority of simulated medical devices and objects located around the mean with few located in variance regions. In some embodiments, the processor determines the best scenario describing the environment, and hence localization of the medical device, from the ensemble based on information collected by sensors of the medical device. At different time points, such as different work sessions, the information collected by sensors may be slightly different and thus a different scenario of any of the feasible scenarios of the ensemble may be determined to be a current localization of the medical device.

Some embodiments use a statistical ensemble to represent multiple possible locations of the medical device as it moves within the surroundings. In some embodiments, the statistical ensemble collapses to at least one possible location of the medical device each time a measurement is taken. Some embodiments evolve the statistical ensemble over time according to an equation of motion of the medical device. Some embodiments infer a location of medical device by: determining a probability of the medical device being located at different possible locations within the surroundings based on the at least one measurement; inferring the location of the medical device based on the probability of the medical device being located at different possible locations within the surroundings, wherein the location of the medical device is inferred at intervals of a predetermined travel distance of the medical device or when the certainty of the location of medical device is below a predetermined threshold.

In some embodiments, the map is divided into small, medium and large tiles. For example, each large tile of the map is divided into a number of smaller tiles. This concept is similar to multiple resolution maps. In a multiple resolution map, the large tile the medical device is positioned on is determined, then the small tile from a subset of tiles that form the large tile the medical device is positioned on is determined. Some embodiments determine one or more tiles the medical device is positioned on with a certain probability. In embodiments, tile size is determined based on a need for performing timely control measures. In one example, the desired probability of the one or more tiles on which the medical device is positioned is 95%. Depending on measurement noise and reading accuracy, a certain minimum number of readings (n) are required. To solve an equation with n inputs, a certain amount of computational time is required. However, during the certain amount of computational time the medical device moves onto another tile. As a result, the objective of having a control measure based on tile size is not met. A constraint of accurate estimation (e.g., maximum tile size) is a contribution to the estimation being obsolete by the time the estimation is completed. Even in tightening the constraint and using a smaller tile size, the medical device leaves the tile faster while more time is required to estimate which tile the medical device was positioned on at the time the process of estimation started. Rather than reducing the tile size or tightening the constraints, constraints are loosened, requiring a certain probability (e.g., %95) of the tile on which the medical device is positioned, and a larger tile is used. Assuming measurement accuracy and noise are the same as the previously described scenario, a smaller number of readings, and therefore, a smaller number of equations and computational time are required to estimate to localize the medical device on a larger tile. With less computational time passing from a beginning of the estimation process until the output estimate, the medical device moves less and the estimation is more relevant. It is more likely the medical device is still positioned on the estimated tile at an end of the estimation process in a case where tiles are larger and medical device speed is constant. However, knowing the tile on which the medical device is positioned does not fully solve the problem but is a step towards the solution. Though a smaller number of inputs are required for estimating a location of the medical device, the inputs are chosen intelligently. Readings that fit a certain criterion of being different from others are selected, thereby preventing a waste of computational time on counting redundant measurements.

Once the large tile the medical device is positioned on is estimated, new incoming measurements are used to determine which small tile from a set of small tiles the medical device is positioned on. New incoming measurements are compared against observation of the surroundings corresponding with different small tiles to determine which observations fits well with the new incoming measurement. With a simple search in a small state space, a location of the medical device t within the small tile map is determined. This second step may be performed using multiple approaches, such as a statistical ensemble, simulation method, or search. In one instance, a particle filter method imagines a number of medical devices, each medical device positioned on a different small tile and each observing a respective map corresponding with their location. Multiple particles are carried forward, reducing a number of particles needed. In another instance, a simple search or comparative validation continuously localizes the medical device for a small tile criteria of a set of tiles forming a larger tile medical device is estimated to be positioned on (determined in the first step). As such, in some embodiments, a multivariate estimation problem with highly dense data points necessary to meet a high certainty/accuracy requirement is decomposed to a lower resolution requirement in a fort step followed by a comparative search to achieve the required resolution. A coarse to fine localization allows for quick localization with minimal computation time and refinement after. This decomposition is not specific to a grid map localization and further allows multithreading.

In yet another example, a RGB camera is set up with a structured light such that it is time multiplexed and synched. For instance, the camera at 30 FPS may illuminate 15 images of the 30 images captured in one second with structured light. At a first timestamp, an RGB image may be captured. In one time slot, objects are detected as features based on sensor data. In a next time slot, the area is illuminated and L2 norm distances are extracted to a plane. With more sophistication, this may be performed with 3D data. In addition to the use of structured light in extracting distance, the structured light may provide an enhanced clear indication of objects. For instance, a grid like structured light projected onto an object provides enhanced indications. The illumination and depth may be used to keep the medical device localized or help regain localization in cases where image feature extraction fails to localize the medical device.

In some embodiments, an image may be segmented to areas and a feature may be selected from each segment. Some embodiments use the feature in localizing the medical device. In embodiments, images may be divided into high entropy areas and low entropy areas. In some embodiments, an image may be segmented based on geometrical settings of the robot. Various types of image segmentations may be used. For instance, image segmentation for feature extraction based on entropy segmentation, exposure segmentation, and geometry segmentation based on geometrical settings of the medical device. Some embodiments may extract a different number of features from different segmented areas of an image. Some embodiments dynamically determine the number of features to track based on a normalized trust value that depends on quality, size, and distinguishability of the feature. For example, if the normalized trust value for five features are 0.4, 0.3, 0.1, 0.05, and 0.15, only features corresponding with 0.4 and 0.3 trust values are selected and tracked. In such a way, only the best features are tracked.

Some embodiments execute a search to determine a location of the medical device, wherein a number of distances to points visible to the medical device may be chosen to be searched against the map to locate the medical device. The denser the population of distances, the higher the confidence, however, more computational resources become at stake. A low resolution search based on a subset of distances may initially be executed to determine areas with a high probability of matching that may be searched in more detail during a next step.

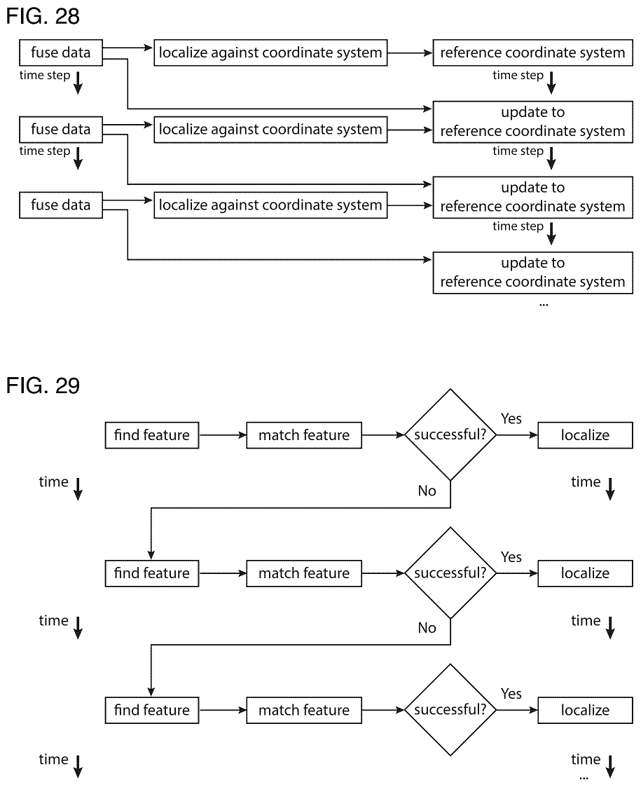

In some embodiments, a first sensor captures depth and a second sensor captures an image. Using depth data and image data, each pixel or group of pixels are associated with a depth value. In some embodiments, image data and depth data are combined to form a map wherein each pixel is associated with a depth value in a particular frame of reference. Some embodiments localize the medical device depth-wise and pixel-wise in a vertical direction and horizontal direction. In some embodiments, multiple camera and depth measuring units simultaneously capture images and depth information for generating a high dimension map for high dimension localization. When multiple devices capable of high dimensional mapping and localization are used in combination for mapping and localization, a hyper dimensional map is created and evolves over time. FIG. 27 illustrates an example of a process for localization of the medical device, wherein data from two sensor types are fused, from which a position of the medical device is inferred and a map is determined. The process then checks for blind spots, if there are no blind spots the medical device continues movement. If there is a blind spot the medical device slows down such that the blind spot is eliminated and the blind spot is filled in from inferred information. FIG. 28 illustrates an example of a process for localizing the medical device, wherein sensor data is fused, from which the medical device localizes against a coordinate system and updates a reference coordinate system. The process is continuously repeated. FIG. 29 illustrates an example of a process for localizing the medical device, wherein a feature is extracted from captured sensor data and an attempt to match the feature to known features within an environment is made. If there is a match, the medical device is localized, and if not, the process is repeated.

Some embodiments include sensors capturing measurements of the surroundings, finding a match between currently observed sensor data and a premade representation of the surroundings, localizing the medical device within the surroundings and a digital representation of the surroundings.

Global localization techniques may be enhanced to accommodate scenarios with a lack of landmarks or matches between currently observed data and previously observed data. When data has a lack of landmarks or a match between currently observed landmarks and landmarks in the saved map cannot be found, it is concluded that the medical device is in a new environment and a new map is created. The new map may be temporary and later merged with an older saved map. Matches are constantly checked for and upon finding a match, the sensed data is stitched with the saved map at overlapping points. However, when a previously detected feature is undetected initially but detected later, appending an already discovered area to the map causes an invalid extension to the map.

During operation of the robot, streams of images coming in may suffer from quality issues arising from a dark environment or relatively long continuous stream of featureless images arising due to a plain and featureless environment. Some embodiments may prevent the SLAM algorithm from detecting and tracking the continuity of an image stream due to the FOV of the camera being blocked by some object or an unfamiliar environment captured in the images as a result of moving objects around, etc. These issues may prevent closing the loop properly in a global localization sense. Therefore, the processor may use depth readings for global localization and mapping and feature detection for local SLAM or vice versa. It is less likely that both sets of readings are impacted by the same environmental factors at the same time whether the sensors capturing the data are the same or different. However, the environmental factors may have different impacts on the two sets of readings.

In some embodiments, the processor may correct uncertainties as they accumulate during localization. In some embodiments, the processor may use second, third, fourth, etc. different type of measurements to make corrections at every state. For instance, measurements for a LIDAR, depth camera, or CCD camera may be used to correct for drift caused by errors in the reading stream of a first type of sensing. While the method by which corrections are made may be dependent on the type of sensing, the overall concept of correcting an uncertainty caused by actuation using at least one other type of sensing remains the same. For example, measurements collected by a distance sensor may indicate a change in distance measurement to an object, while measurements by a camera may indicate a change between two captured frames. While the two types of sensing differ, they may both be used to correct one another for movement. In some embodiments, some readings may be time multiplexed. For example, two or more IR or TOF sensors operating in the same light spectrum may be time multiplexed to avoid cross-talk. Some embodiments may combine spatial data indicative of the position of the medical device within the environment into a block and may process the spatial data as a block. This may be similarly done with a stream of data indicative of movement of the medical device. Some embodiments may use data binning to reduce the effects of minor observation errors and/or reduce the amount of data to be processed. Some embodiments may replace original data values that fall into a given small interval, i.e. a bin, by a value representative of that bin (e.g., the central value). In image data processing, binning may entail combing a cluster of pixels into a single larger pixel, thereby reducing the number of pixels. This may reduce the amount data to be processed and may reduce the impact of noise.

Multi-type landmark extraction, observation of a sophisticated type of landmark, may be used sparsely and at intervals to determine which group of clusters of primal landmarks to search for to find a match. In some embodiments, images are clustered to reduce the search domain. In some embodiments, content-based image retrieval is used to match similar images in a large database. Similarity may be defined based on color, texture, objects, primal shapes, etc. In some embodiments, a query image is captured in real-time and the processor determines which image in a database the query image correlates to. In some embodiments, a vector space model is used to create visual words from local descriptors, such as SIFT, wherein a visual vocabulary is the database of previously captured images. A subset of images may include objects of interest or a detected object. In some embodiments, images are indexed in a database. The localization methods described herein are not restricted to one implementation of SLAM. For example, a first type of landmark may be a visual type (e.g., primal shape in an image), a second type of landmark may be a map type, a third type of landmark may be an object detected in image, etc.

Landmarks may be extracted from a laser scan or camera data and compared against any number of landmarks that have been observed a sufficient amount of times to determine if the newly observed landmarks are newly observed landmarks or previously observed and identified landmarks. When a landmark observation undergoes probabilistic criteria and is categorized as a previously observed and identified landmark, the landmark is categorized in a same category set as the previously observed landmark to which it matches. The category set then has one more variation of the landmark wherein the observation may be captured from a different angle, under different lighting conditions, etc. When no match to a previously observed landmark is found, the observed landmark becomes a first element of a new category set. As more observations are collected, a number of elements within the category set increases. When a number of elements in a category set is large enough, the landmark is considered highly observable. The larger the category set is, the more important the landmark is. In some embodiments, only category sets with a number of elements above a predetermined threshold are used for comparison against a landmark observation when determining the category. When a landmark is observed, a position and orientation of the medical device in relation to the landmark with a probability of error is determined. With advancement of object recognition and algorithms, landmarks are identified as context associated with landmarks provides useful information. A landmark database may include labels associated with different types of objects. In some embodiments, labels are probability based, wherein upon repeated observation and recognition of a same object type the probability of the object type increases to reach a confidence level. As identified objects may not constitute a sufficient number of landmarks, the identified landmarks may be used in addition to more primitive landmarks in legacy visual SLAM systems. While not all the landmark objects may be identified to have a human classification associated with them, the mere process of extracting a more sophisticated shape than just an arc, circle, blob, line, edge provides significantly more certainty upon repeated observation. For instance, a primal shape, such as a blob, can be easily mistaken. Even in a case where the classification algorithm fails to identify the particular object type of the object, extraction of such a sophisticated shape without the particular object type still provides a degree of certainty when it is observed again. A downside of extracting an object without any further knowledge on context is repetition throughout the surroundings (e.g., a molar on a right side versus a molar on a left side).

Some embodiments use EKF to estimate a position and an orientation of the medical device from contextual landmarks. The position and orientation of the medical device is iteratively updated, based on displacement of the medical device, new observations of previously observed contextual landmarks, and new observation of previously unobserved contextual landmarks. When a landmark is categorized, the landmark may have a hierarchical importance value. For example, a primitive shape, such as an edge, a line, a blob, or an arc, may be found more often and at shorter distances while more sophisticated objects may be detected less frequently but are distinct. Covariance of two variables provides an indication of an existence or strength of correlation or linear dependence of the variables. A covariance matrix in the current state of the art provides a single type of landmark, while herein multiple types of landmarks (e.g., primal landmarks, sophisticated landmarks) are provided. In the proposed method, covariance between a medical device state and a first type of landmark is different and distinguished from a covariance between a medical device state and second type of landmark. While a first type of landmark may be more densely present or frequently observed in the surroundings in comparison to a second type of landmark that may be scarcer or difficult to detect, the second type of landmark provides higher confidence and helps close the loop. There are many primal landmarks, some semi-sophisticated landmarks, and few sophisticated landmarks. The more sophisticated landmarks provide higher confidence during SLAM. There is no requirement of a specific number of primal landmarks per sophisticated landmark.

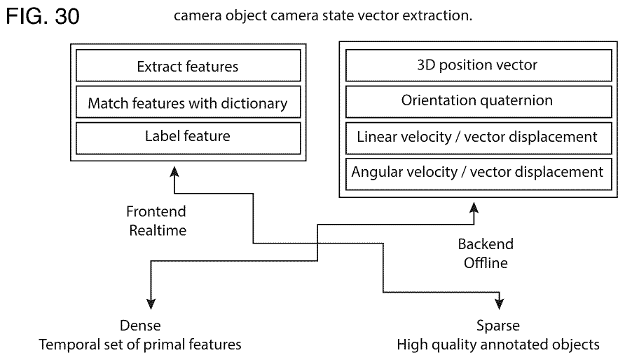

FIG. 30 illustrates a diagram of camera object and camera state vector extraction. In the frontend, features are extracted and matched with features within a dictionary, and based on a match, the features are labelled as an object type. This provides sparse, high quality annotated objects. In the backend, a 3D position vector and orientation quaternion are used in determining linear and angular velocity and displacement. This provides a dense, temporal set of primal features.

In some embodiments, object recognition is used as a secondary and more reliable landmark to localize against, in particular stationary and structural objects. Identification of these objects relies on detecting a series of primal features in a specific arrangement. In some embodiments, a structure is first identified, then labeled. Even if the structure is labeled incorrectly, the mere identification of the features in a specific arrangement may still be used as a landmark for localization as localization is solely based on recognizing the particular features in the specific arrangement, the label being unnecessary. For example, as long as the structure is captured in an image and a localization algorithm detects the features in a specific arrangement forming the structure, a loop may be closed. Labeling the structure depends on existing data sets, examples of the structure, lighting conditions, and such. A user may label examples of structures captured in images using the application to improve local recognition success results. Structures labelled by the user may be given more weight. User labelling may also improve global recognition success as users collectively provide a large amount of labelling, providing both labeling volume and labeling diversity (important for objects and situations that are very difficult to artificially stage and ask operators to label).

For both structural object identification and labeling, illumination, depth measurement, and a sequence of images are useful. Illumination is helpful as the addition of some artificial light to the environment reduces the impact of the ambient environment. Illumination may be employed at intervals and illuminated images may be interleaved with non-illuminated images. Depth measurements may be captured with a depth camera, built-in TOF sensors, a separate TOF coupled to the robot, a structural light, or a separate measurement device for depth based object recognition. A sequence of images may also improve object identification and labeling success rate. For example, an image with illumination followed by an image without illumination may provide better insight than images without any illumination. A sequence of two, three, five, six, ten or another number of frames captured one after another as the medical device is moving captures the structure of the object from slightly different angles. This provides more data, thereby reducing false positives or false negatives. The number of image frames used may be fixed in a sliding window fashion or dynamic in a dynamic window fashion.

In some embodiments, clustering and K-means algorithm are used to group similar images together. Similarity may be based on a gray scale image or may be based on type 1 features (primal structures) or type 2 features (sophisticated structures). In either case, an estimate of localization and organization of images in a grid reduces the search space drastically. Inside the grid or a group of grids, clustering may be used to further organize the proposal domain into a structured system wherein creative search methods may be used to match a current run input with pre-saved data (such as an image). Some embodiments use search methods implementing a K-d tree or a Chou-Liu hierarchy. As opposed to prior art that use a simple tree, a two (or more) type feature detection may interleave feature types or create separate trees for each feature type with sparse or dense association with other feature types. When a search is performed for a type 2 feature the search domain is small, however, the search phrase is complex and comprises a structure formed of primal features. For a match to occur, a more sophisticated criterion is required. For a type 1 feature, the search domain is small and the term is simple. Several matches are likely to be found quickly, however, false positives are more likely. An example data set may be previously generated and available or may be built. The example data set does not have to be labeled, although a labeled data set may be used.

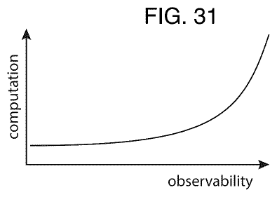

In embodiments, the different types of landmark may have geometric relations, topological relations, or graph relations with one another (direct or indirect). The relations between different types of landmarks may be perceived, extracted gradually, or may remain unknown to the SLAM algorithm. It is not necessary for all relations to be discovered for SLAM. As relations are discovered they are used where beneficial and when undiscovered, the SLAM continues to operate under circumstances of partial observability. The SLAM algorithm is always in a state of partial observability, even as more observations are made and relations are inferred. FIG. 31 illustrates a correlation between observability and computation needs. A real-time system suffers as constraints are added, especially during occasions of peak processing. As a result, some embodiments process basic SLAM at a low level. In some embodiments, features are extracted to provide a degree of sparsity rather than directly tracking a point cloud or pixels. For example, RANSAC extracts a line or edge from a point cloud and Haar extracts a line or edge from an image. In embodiments, pose estimation and tracking are executed serially or in parallel. Multi-threading is most advantageous when a multicore architecture is used.

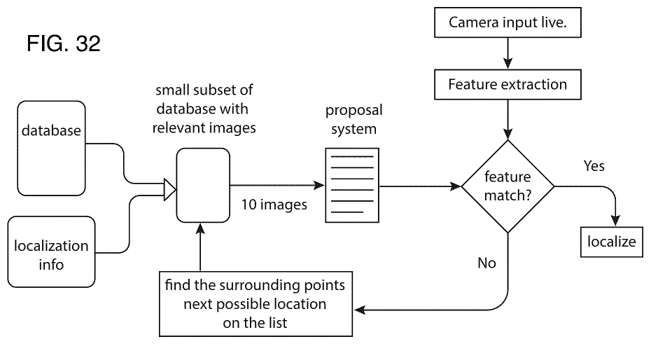

When a location of the medical device is approximately known, the algorithm does not have to search through all images within the database to localize the medical device at a next time step. To match a feature observed in a newly captured image with the environment, the algorithm searches for the feature within a subset of images associated with (x, y, Θ). In some cases, the database may not have entries for all possible poses or cells within the map. In such cases, ML algorithms select nearest neighbors that have a highest chance of matching the observed feature. Near may be defined based on Euclidean or Mahalanobis or is defined probabilistically. For a highest chance of success in matching the feature, matches occur in parallel, wherein a group of images are matched against another group of images. The algorithm may sort through a database and propose a set of potential matching images. FIG. 32 illustrates an example of a process of localizing the medical device. Based on localization information, a small subset of the database with relevant images are provided to the proposal system which sorts through the subset and proposes a set of potential matches. Features are extracted from images captured by a live camera and compared against the images with a high likelihood of matching. If there is a match, the medical device is localized, otherwise another subset of the database corresponding to areas close to the first considered subset are provided to the proposal system to find a match.

In some embodiments, the database is not pre-sorted or has inaccuracies. K-means clustering may be used to create K clusters, wherein K is a possible number of poses of the medical device (including coordinate and heading). Sum of squared distance may be used to create an elbow curve. Hierarchical clustering may be used to create a dendrogram and identify distinct groups. In some embodiments, the first two images close to one another (based on a distance of their histograms or plotted pixel densities), wherein distance may be Mahalanobis or Euclidean, are representative of a cluster. In embodiments, a vertical height of a dendrogram represents such distance.

Clustering may be used to organize previously captured images and/or depth readings when creating a proposal of images to match a new reading against. Clustering comprises an assigning a label to each data element when a previous labeling is non-existent. In some embodiments, clustering is combined with chronicle labeling of a previous session or a current session. For instance, X={x₁, x₂, . . . xᵢ, . . . xₙ} is a set of N data elements and cluster C₁, C₂, . . . Cₖ is a subset of set X, wherein each C is disjoint from the others and has elements presented by one of the elements in the subset of set X. Algorithms, such as Lloyd's algorithm, may be used to cluster a given set of data elements. Some embodiments use soft K-means clustering, wherein a soft max function is used. Once soft assignments are computed, centroids may be found using a weighted average. Some embodiments use a latent variable model to observe hidden variables indirectly from their impact on the observable variables. An example is a Gaussian mixture model wherein an expectation-maximization is employed to compute the MLE.

In some embodiments, active illumination helps with feature association, whereas in embodiments with no illumination, image associations between two time steps relies on brute-force matching and sorting the matches based on a metric such as Euclidean distance and hamming distance. As an alternative to active illumination, the search domain may be reduced based on real-time localization information obtained from a point cloud/distance data or a previous run. Motion estimation may also be used to reduce a search domain. In some embodiments, methods such as clustering are used to organize images. Methods such as ICP and PnP, discussed in detail elsewhere herein, may be used. In some embodiments, a mono-camera setup is used to add an active illumination point to enhance and ease key point extraction by reducing the search space. A neighborhood surrounded by the illumination may be used to extract descriptors, which often demands high computational resources, and in the prior art, may be discarded. However, with the number of key points heavily reduced due to a high reliability of key points (through the use of illumination), descriptors may be preserved. Further, active illumination improves sparse optical flow algorithms such as Lucas-Kanade that traditionally suffer from constant grayscale assumption. In a preferred embodiment, an effect of ambience light is automatically reduced as active illumination impacts image brightness. In some embodiments, a coarse to fine optical flow is traced in a pyramid scheme of image arrangement.

In a monocular observation setup, a pose graph is created at each coordinate where an observation is made and an essential matrix is synthesized to relate observations at each coordinate. For each straight trajectory with an approach to a feature, temporal triangulation provides consecutive readings that may be tracked as an optical flow. In some embodiments, RANSAC is used to find a best perspective transform when corresponding sets of points are ambiguous, imperfect, or missing. When coplanar points are not available, the essential matrix may be used to associate any sets of points in one image to another image as long as a same camera is used in capturing the images and intrinsic matrices are constant. Fundamental matrices may be used where intrinsic matrices are not constant.

Maximum likelihood estimation is often used in solving non-convex, non-linear problems, such as pose graph estimation (e.g., for camera or LIDAR) or bundle adjustment. Usually, a global optimality is unguaranteed unless the observation noise or motion recording is kept on strict check using, for example, Newtonian trust region and equivalent methods. These methods apply to 3D or 2D point swarms, image streams, and a synchronized RGB+D input or object label placement on an already synchronized spatial map.

Some embodiments may use at least some of the methods, processes, and/or techniques for determining a location of the robot described in U.S. Non-Provisional patent application Ser. Nos. 16/297,508, 16/509,099, 17/494,251, 17/344,892, 17/670,277, 17/990,743, 15/425,130, 15/955,480, 16/554,040, and 18/503,093, each of which is hereby incorporated herein by reference.

In embodiments, processing occurs on a same MCU that sensing and actuating occur on, eliminating the physical distance between the point of data collection and data processing. Some embodiments implement a method for reducing the computational intensity of SLAM by use of a microcontroller or MCU for information processing at the source instead of a CPU that must be distanced from corresponding sensors and actuators. In some embodiments, all processes run on a single MCU. In some embodiments, the same single MCU controls SLAM, sensing and actuation, applications that control components of the medical device or the robot operating the medical device.

Some embodiments use a MCU (e.g., SAM70S MC) including built in 300 MHz clock, 8 MB Random Access Memory (RAM), and 2 MB flash memory. In some embodiments, the internal flash memory may be split into two or more blocks. For example, a lower block may be used as default storage for program code and constant data. In some embodiments, the static RAM (SRAM) may be split into two or more blocks. In some embodiments, all tasks are scheduled to run on the MCU. Information is received from sensors and is used in real time by algorithms. Decisions actuate actuators without buffer delays based on the real time information. Examples of sensors include, but are not limited to, IMU, gyroscope, OTS, depth camera, obstacle sensor, acoustic sensor, camera, image sensor, TOF sensor, TSOP sensor, laser sensor, light sensor, electric current sensor, optical encoder, accelerometer, compass, speedometer, proximity sensor, range finder, LIDAR, LADAR, radar sensor, ultrasonic sensor, piezoresistive strain gauge, capacitive force sensor, electric force sensor, piezoelectric force sensor, optical force sensor, capacitive touch-sensitive surface or other intensity sensors, GPS, etc.

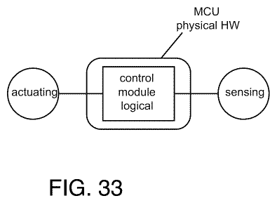

In embodiments, the MCU reads data from sensors, selects a mode of operation, automatically turns various components on and off or per user request, receives signals from remote or wireless devices and sends output signals to remote or wireless devices using Wi-Fi, radio, etc., self-diagnoses the medical device or the robot, operates a PID controller, controls pulses to motors, controls voltage to motors, controls battery charging, etc. FIG. 33 illustrates an example of an MCU and tasks executed by the MCU.

Some embodiments use at least some components, methods, processes, and/or techniques for processing data required in operating the medical device or the robot operating the medical device described in U.S. Non-Provisional patent application Ser. Nos. 17/494,251, 17/344,892, 17/670,277, and 17/990,743, each of which is hereby incorporated herein by reference.

The methods and techniques described herein may be implemented as a process, as a method, in an apparatus, in a system, in a device, in a computer readable medium (e.g., a computer readable medium storing computer readable instructions or computer program code that may be executed by a processor to effectuate robotic operations), or in a computer program product including a computer usable medium with computer readable program code embedded therein.

While this invention has been described in terms of several embodiments, there are alterations, permutations, and equivalents, which fall within the scope of this invention. It should also be noted that there are many alternative ways of implementing the methods, devices and apparatuses of the present invention. Furthermore, unless explicitly stated, any method embodiments described herein are not constrained to a particular order or sequence. Further the Abstract is provided herein for convenience and should not be employed to construe or limit the overall invention, which is expressed in the claims. It is therefore intended that the following appended claims to be interpreted as including all such alterations, permutations, and equivalents as fall within the true spirit and scope of the present invention.

In block diagrams, illustrated components are depicted as discrete functional blocks, but embodiments are not limited to systems in which the functionality described herein is organized as illustrated. The functionality provided by each of the components may be provided by specialized software or specially designed hardware modules that are differently organized than is presently depicted; for example, such software or hardware may be intermingled, conjoined, replicated, broken up, distributed (e.g. within a data center or geographically), or otherwise differently organized. The functionality described herein may be provided by one or more processors of one or more computers executing specialized code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation.

1. A method for localizing an electronic device, comprising:
capturing data of surroundings of the electronic device with at least one sensor of the electronic device; and
inferring a location of the electronic device based on at least some of the data of the surroundings, wherein inferring the location of the electronic device comprises:
determining a probability of the electronic device being located at different possible locations within the surroundings based on the at least some of the data of the surroundings; and
inferring the location of the electronic device based on the probability of the electronic device being located at different possible locations within the surroundings.

2. The method of claim 1, wherein a statistical ensemble is used to represent multiple possible locations of the electronic device as the electronic device moves within the surroundings and the statistical ensemble reduces to at least one location when the at least one measurement is captured.

3. The method of claim 1, wherein the method further comprises:
obtaining depth data, wherein:
the depth data indicates a distance from a position of the handheld device to a surface within the surroundings;
the depth data indicates a direction in which the distance is measured; and
the depth data indicates the distance and the direction in a frame of reference of the electronic device.
